(19) United States
(12) Patent Application Publication
(10) Pub. No.: US 2015/0059746 A1
(43) Pub. Date: Mar. 5, 2015

(54) METHOD AND APPARATUS

(71) Applicant: VECTURA LIMITED, CHIPPENHAM (GB)

(72) Inventor: Matthew Green, Chippenham (GB)

(73) Assignee: VECTURA LIMITED

(21) Appl. No.: 14/387,629

(22) PCT Filed: Mar. 28, 2013
(86) PCT No.: PCT/GB2013/050847

§ 371 Date: Sep. 24, 2014

(30) Foreign Application Priority Data
Mar. 30, 2012 (GB) 1205632.1

(51) Int. Cl. B01F 11/02; A61J 3/02; A61K 9/00; B02C 19/06; A61M 15/00

(52) U.S. Cl. CPC B01F 11/02; B02C 19/06; A61M 15/00; A61K 9/0075; A61J 3/02; B01F 2215/0032; A61M 2202/064; A61M 2207/10
USPC 128/203.15; 366/108; 241/5; 241/25; 53/428; 427/2.14; 514/171; 514/312; 514/291; 514/305; 514/424

(57) ABSTRACT

A method is disclosed for making a pharmaceutical composition for pulmonary administration, the method comprising a step in which an inhalable pharmaceutically active material is acoustically blended in a resonant acoustic blender. The invention also relates to compositions for inhalation prepared by the method.

### BACKGROUND

The present invention relates generally to the field of mixing, specifically acoustic mixers for mixing powders. The apparatus is particularly suited for efficiently blending powders to be used in the ceramics, propellant, chemicals, food and beverage or cosmetics industries. More particularly, the present invention is directed to the field of pharmaceuticals, specifically the field of inhalation.

Inhalation represents a very attractive, rapid and patient-friendly route for the delivery of systemically acting drugs, as well as for drugs that are designed to act locally on the lungs themselves. It is particularly desirable and advantageous to develop technologies for delivering drugs to the lungs in a predictable and reproducible manner.

The key features which make inhalation a useful drug delivery route are: rapid speed of onset; improved patient acceptance and compliance for a non-invasive systemic route; reduction of side effects; product life cycle extension; improved consistency of delivery; access to new forms of therapy, including higher doses, greater efficiency and accuracy of targeting; and direct targeting of the site of action for locally administered drugs, such as those used to treat lung diseases.

However, the powder technology behind successful dry powders and dry powder inhaler (DPI) or pressured metered dose inhalers (pMDI) products remains a significant technical hurdle to those wishing to succeed with this route of administration and to exploit the significant product opportunities. Any suitable formulation must have properties that allow for the manufacture and metering of the powders, provide reliable and predictable resuspension and fluidisation, and avoid excessive retention of the powder within the dispensing device. One way of obtaining a resuspension and fluidisation involves mixing or blending of the formulations to be used in DPIs or pMDIs.

The mixing or blending of powders involves agitation resulting in the distribution of either heterogeneous or homogeneous particles to form the final formulation. Mixing processes are called upon in an attempt to effect a uniform distribution of particulates such as drug particles over a carrier particle.

Traditionally, mixing can be achieved in a variety of ways. Firstly, by a rotating shaft mounted impeller which is immersed in the fluid mixture. Secondly, by tumbling the fluid mixture in a container vessel, or finally by vibrating the fluid mixture. Mixing may be continuous or intermittent.

Equipment, such as tumblers and tube blenders are well-used in industry often achieving blend uniformity after prolonged blending times. Unfortunately, segregation of these blends can readily occur especially during subsequent blend handling or transfer. The negative effects of segregation can include: uneven particle size or drug distribution, decreased flowability, reduced performance as well as changes in blend colour, taste, or appearance. Segregation is particularly prevalent when particles separate due to differences in their size, shape, or density. A further criticism levelled at traditional blending procedures, especially impeller blending processes, is that a substantial proportion of the blend is lost to the internal surfaces. This is a particular disadvantage for blends that contain expensive drugs. Traditional impeller processors are also known to generate heat within the blend, which may adversely affect the blend characteristics.

In contrast to impeller blending processes, a conventional vibrational mixer, does not contain moving parts that generate a homogenous blend. Vibratory machines have been well known for many years for a large number of different uses, including screening and compacting of concrete mixtures and powders, tamping of soil and asphalt, shaking-out of molds and casting, crushing, milling and mixing of powders. Such machines further find application in a plurality of fields, including the construction industry, manufacture of building materials, processing of raw materials, mining, metallurgy, mechanical engineering, foundry associated applications, manufacture of ceramics and powders, the food industry, pharmaceuticals and chemicals.

Throughout all the processes mentioned above, amplitude and frequency are the parameters of importance. It is understood for some time, that mixing the blend at the natural resonant frequency of the mixer should be avoided in order to avoid associated wear of the mechanisms.

A major problem experienced by formulators is that uniform blends often take time to generate. This approach it is often associated with problems such as poor blend uniformity and undesirable heating of the constituent parts. Formulators face a delicate balance because over processing the formulation may change the blend dispersion characteristics thereby creating unwanted inter-batch variability. Conversely, under-processing may lead to the generation of API “hotspots” which may not be detected by conventional blend uniformity tests. This is further complicated in the field of inhalation where not only is a uniform blend a prerequisite of a suitable formulation, but the dissociation of the active from the carrier must take place at a specific time in order to deliver a therapeutic dose to the patient. Uniform blends can be achieved using conventional machines but this often involves high energy blending and mixing procedures with rapid rotation speeds that impart undesirable effects to the powder such as, for example, heat, static or undesired milling of the particles.

In summary, the background art does not teach a system suitable for producing formulations suitable for inhalation. What is needed is a rapid method for uniformly mixing particulates in a manner that can be varied whilst still maintaining the physical structure of the fragile drug and excipient materials within the pharmaceutical formulation.

### SUMMARY OF THE INVENTION

In view of the problems outlined above, the present application teaches the use of a resonant acoustic mixer for mixing of powders with advantageous blend homogeneities and aerosol performance.

The purpose of the invention is to provide a method of intimate processing of, for example, a plurality of fluids. These fluids may include liquid-liquid, solid-solid, liquid-solid or more than two fluid phases. One application is the mixing and dispersion of solids, in particular small particles. Other applications include preparing emulsions for chemical and pharmaceutical applications, accelerating physical and chemical reactions, for example biological reactions such as enzymatic processes, and suspending fine particles in fluids. The fluids referred to above may or may not include entrained solid particles. One application is the mixing and dispersion of fluids, for example solids, in particular small solid particles.

The present invention provides a method for mixing materials which afford minute control over mixing in a wide range of applications. The range of applications extends from bench scale formulations (up to 450 g) to large scale manufacture of pharmaceuticals (up to 420 kg). In one embodiment, the present invention provides a vibration mixer, driven by an electronically controllable motor or motors, adapted to allow control of the mixing process.

Yet another embodiment of the invention is a process to facilitate mixing by a selected frequency, amplitude or acceleration. Another embodiment of the invention is to disperse fine particles in a uniform manner throughout the formulation blend.

In one embodiment said composition comprises a plurality of particles and said mixing step further comprises exposing said composition to a vibratory environment that is at a frequency between about 15 Hertz to about 1,000 Hertz and at an amplitude of between about 0.01 mm to about 50 mm thereby achieving micromixing of said composition.

A system and process for the application of acoustic energy to a reactor volume that can achieve a high level of uniformity of mixing is disclosed. The “micromixing” that is achieved and the effects in the combinations of frequency ranges, displacement ranges and acceleration ranges disclosed herein produce very high-quality blends, as defined by acceptable blend uniformity and constituent parts which exhibit improved physical character, for example aerosol performance and/or stability. This is especially noticeable when preparing delicate carrier systems.

The method disclosed herein can be practiced with the systems disclosed herein and with single mass vibrators, dual mass vibrators, and piezoelectric and magnetostrictive transducers.

Although some embodiments are shown to include certain features, the applicant(s) specifically contemplate that any feature disclosed herein may be used together or in combination with any other feature on any embodiment of the invention. It is also contemplated that any feature may be specifically excluded from any embodiment of an invention.

The invention relates, in one aspect, to a method for making a pharmaceutical composition, the method comprising a step in which particles of pharmaceutically active material are acoustically blended in the presence of particles of an excipient material.

A pharmaceutically active material, also referred to as active pharmaceutical ingredient (API), is the substance in a pharmaceutical composition that is biologically active. The distinction between an API and excipient can be determined by referring to pharmaceutical reference literature. Furthermore, an inhalable API must also have particle size distribution wherein D₁₀≦6 μm, D₅₀≦7 μm and D₉₀ 10 μm.

Without wishing to be bound by theory, the method of acoustically blending according to the present application provides for a homogenous mixing of material by an acoustic mixing method. The formulation is subjected to vibration at an amplitude and frequency that causes resonance of the particles within the formulation. When focused on a formulation, the acoustic energy converts into particle kinetic energy which, in isolation, is relatively insignificant. When the acoustic energy is focused on a population of particles the pockets of energised particles affected rapidly mix with surrounding particles. This resonance causes macroscopic and microscopic turbulence within the blend enabling uniform mixing. Mixing using an acoustic blender is therefore quickly achieved without the use of impellors, blades, rotors, paddles or rotation of the containing vessel. Homogenous mixing of the pharmaceutical composition can be determined by a percentage coefficient of variation that is less than about 5%.

Similarly, when a suspension or bi-phasic liquid formulation is subjected to vibration at an amplitude and frequency that causes resonance of the liquid formulation. When focused on the liquid formulation, the acoustic energy affects the surfaces of the liquids causing them to ripple. As the energy is increased the ripple becomes greater until protrusions or invaginations occur at the liquid surface. Eventually these protrusions are so extensive that they break away from the liquid formulation entirely to join the other liquid phase and thereby create a new liquid-liquid surface and so the process is repeated until a distinct liquid-liquid surface no longer exists but at a macroscopic level the “bi-phasic” liquid formulation has now become uniform in appearance. Mixing is therefore quickly achieved without the use of impellors, blades, rotors or paddles.

### DETAILED DESCRIPTION OF INVENTION

An embodiment of the invention is to facilitate acoustic mixing of two or more solids. Another embodiment of the invention is to facilitate acoustic mixing of one or more solids and one or more gases. Another embodiment of the invention is to facilitate acoustic mixing of one or more solids with one or more liquid particles. A further embodiment of the invention is to facilitate acoustic mixing of one or more solid with one or more liquid particles with one or more gases.

Mixing gram (g) to kilogram (kg) amounts of pharmaceutical composition is contemplated according to all embodiments. Mixing milligram (mg), nanogram (ng) and smaller amounts of pharmaceutical composition are impractical and therefore not suitable due to loss of the constituents to the vessel wall. Likewise, mixing tonnes of pharmaceutical composition are also impractical because of the difficulties associated with routinely obtaining homogenous blends. Blend homogeneity is particularly important in the field of pulmonary drug delivery.

Solids are mixed by adding acoustic energy so that micromixing is achieved. A vibratory environment operating at a frequency between about 15 Hz to about 1,000 Hz with an amplitude between about 0.01 mm to about 50 mm provides the necessary acoustic energy required to mix solids. The size of the solids can be nano-sized to much larger particles, for example micrometers. The acoustic energy provided to the particles directly acts on the formulation to produce mixing. Other processes use components such as propellers to produce fluid motion through eddies which then mix the media. These eddies are dampened by the media and thus the mixing is localized near the component creating them, for example the blades, rotors or paddles. Acoustic energy supplied to the media is not subject to the localization of input mentioned above because the entire mixing vessel volume is subject to the energy at the same time.

Specific frequency ranges for operating the acoustic blender include from about 5 Hz to about 1,000 Hz, preferably 15 Hz to about 1,000 Hz, more preferably 20 Hz to about 800 Hz, more preferably 30 Hz to 700 Hz, more preferably 40 Hz to 600 Hz, more preferably 50 Hz to 500 Hz, more preferably 55 Hz to 400 Hz, more preferably 60 Hz to 300 Hz, more preferably 60 Hz to 200 Hz, more preferably 60 Hz to 100 Hz, more preferably 60 Hz to 80 Hz, more preferably 60 Hz to 75 Hz, most preferably from about 60 to 61 Hz. The selection of the resonant frequency is the most important criterion because acceleration, amplitude and intensity can be modified accordingly. The selection of less energetic parameters as illustrated in example 10 below will require either extended duration of acoustic blending or the selection of more energetic parameters as illustrated in example 15 below.

Specific time ranges for operating the acoustic blender include from at least 10 seconds, at least 30 seconds, least 1 minute, for at least 2 minutes, for at least 3 minutes, for at least 4 minutes, for at least 5 minutes, for at least 6 minutes, for at least 7 minutes, for at least 8 minutes, for at least 9 minutes, for at least 10 minutes, for at least 11 minutes, for at least 12 minutes, for at least 13 minutes, for at least 14 minutes, for at least 15 minutes, for at least 16 minutes, for at least 17 minutes, for at least 18 minutes, for at least 19 minutes, for at least 20 minutes, for at least 21 minutes, for at least 22 minutes, for at least 23 minutes, for at least 24 minutes, for at least 25 minutes, for at least 26 minutes, for at least 27 minutes, for at least 28 minutes, for at least 29 minutes or for up to 60 minutes or for up to 30 minutes. For the avoidance of doubt, blending periods of less than 30 seconds are less preferred because whilst homogenous blends can be achieved as demonstrated in Example 4 at 10 seconds, they are not routinely achievable as determined by a percentage coefficient of variation that is greater than about 5%. The specific time periods disclosed herein refer to periods in which resonance is imparted to the pharmaceutical composition. It is possible for the resonance blending to be interrupted whilst, for example, content uniformity of the pharmaceutical composition is established. Upon completion of the content uniformity assessment, resonance blending may be resumed. The total duration of resonance blending of the pharmaceutical composition or of its constituent parts will be understood to be the specific time period disclosed herein. It is important to establish once a coefficient of variation of less than 5% has been achieved because acoustic blending should then be stopped, or closely monitored if a coefficient of variation of less than 4%, or less than 3%, or less than 2% or less than 1% is desired. It is possible to impart too much acoustic energy for too long and produce a formulation wherein the blend is not homogenous (due to re-segregation) as determined by a percentage coefficient of variation that is greater than about 5%. Diligent monitoring of the blend's content uniformity during acoustic blending will ensure this (i.e. CV>5%) does not happen.

In one embodiment, a method is disclosed for making a pharmaceutical composition, the method comprising a step in which an inhalable pharmaceutically active material is acoustically blended with excipient material, wherein the acoustic frequency operating range is from 5 Hz to about 1,000 Hz for a period of at least for at least 2 minutes. Preferably, wherein the excipient material comprises lactose.

In one embodiment, a method is disclosed for making a pharmaceutical composition, the method comprising a step in which an inhalable pharmaceutically active material is acoustically blended with excipient material, wherein the acoustic frequency operating range is from 5 Hz to about 1,000 Hz for a period of at least for at least 2 minutes until a coefficient of variation of less than 5% is achieved. Preferably, wherein the excipient material comprises lactose.

In one embodiment, a method is disclosed for making a pharmaceutical composition, the method comprising a step in which an inhalable pharmaceutically active material is acoustically blended with excipient material and additive material, wherein the acoustic frequency operating range is from 5 Hz to about 1,000 Hz for a period of at least for at least 2 minutes. Preferably, wherein the excipient material comprises lactose.

In one embodiment, a method is disclosed for making a pharmaceutical composition, the method comprising a step in which an inhalable pharmaceutically active material is acoustically blended with excipient material and additive material, wherein the acoustic frequency operating range is from 5 Hz to about 1,000 Hz for a period of at least for at least 2 minutes until a coefficient of variation of less than 5% is achieved. Preferably, wherein the excipient material comprises lactose.

In one embodiment, a method is disclosed for making a pharmaceutical composition, the method comprising a step in which an inhalable pharmaceutically active material is acoustically blended with excipient material and magnesium stearate, wherein the acoustic frequency operating range is from 5 Hz to about 1,000 Hz for a period of at least for at least 2 minutes. Preferably, wherein the excipient material comprises lactose.

In one embodiment, a method is disclosed for making a pharmaceutical composition, the method comprising a step in which an inhalable pharmaceutically active material is acoustically blended with excipient material and magnesium stearate, wherein the acoustic frequency operating range is from 5 Hz to about 1,000 Hz for a period of at least for at least 2 minutes until a coefficient of variation of less than 5% is achieved. Preferably, wherein the excipient material comprises lactose.

In one embodiment, a method is disclosed for making a pharmaceutical composition, the method comprising a step in which an inhalable pharmaceutically active material is acoustically blended with excipient material and magnesium stearate, wherein the acoustic frequency operating range is from about 30 Hz to 75 Hz for a period of at least for at least 2 minutes. Preferably, wherein the excipient material comprises lactose.

In one embodiment, a method is disclosed for making a pharmaceutical composition, the method comprising a step in which an inhalable pharmaceutically active material is acoustically blended with excipient material and magnesium stearate, wherein the acoustic frequency operating range is from about 30 Hz to 75 Hz for a period of at least for at least 2 minutes until a coefficient of variation of less than 5% is achieved. Preferably, wherein the excipient material comprises lactose.

In one embodiment, a method is disclosed for making a pharmaceutical composition, the method comprising a step in which an inhalable pharmaceutically active material is acoustically blended with excipient material and magnesium stearate, wherein the acoustic frequency operating range is from about 60 Hz to 75 Hz for a period of at least for at least 2 minutes. Preferably, wherein the excipient material comprises lactose.

In one embodiment, a method is disclosed for making a pharmaceutical composition, the method comprising a step in which an inhalable pharmaceutically active material is acoustically blended with excipient material and magnesium stearate, wherein the acoustic frequency operating range is from about 60 Hz to 75 Hz for a period of at least for at least 2 minutes until a coefficient of variation of less than 5% is achieved. Preferably, wherein the excipient material comprises lactose.

In one embodiment incorporation of a solid into a liquid is enhanced by exposing the solid and liquid to a vibratory environment that is operative to vibrate the combination at a frequency of between about 15 Hz to about 1,000 Hz with amplitude between 0.01 mm to about 50 mm. Incorporation can be so complete it is approaching the theoretical maximum. By placing the fluid and solids in a vibratory environment and, as a result, providing acoustic energy to the media, the effect is to fluidize the mixture. In the process, micromixing is accomplished throughout the vessel while macro-mixing the product. Complete and thorough mixing is accomplished by the use of acoustic energy at previously unachievable solids loadings.

One embodiment of the invention is to facilitate acoustic mixing of two or more liquids, for example two or more miscible liquids (a linctus), or for example two or more non-miscible liquids (emulsions or creams). Another embodiment of the invention is to facilitate acoustic mixing of one or more liquids and one or more gases. Another embodiment of the invention is to facilitate acoustic mixing of one or more liquids with one or more solid particles. A further embodiment of the invention is to facilitate acoustic mixing of one or more liquids with one or more solid particles with one or more gases.

Liquid to liquid mixing is enhanced when a composition that comprises a plurality of liquids is exposed to a vibratory environment that vibrates the composition at a frequency between about 15 Hz to about 1,000 Hz with an amplitude between about 0.01 mm to about 50 mm. Liquids that are not miscible are readily mixed when subjected to this condition. Normal boundary layers which prevent mixing are broken and the liquids are freely and evenly distributed within each other. Micromixing with generation of micron to 100 micron droplets is achieved in this vibratory environment. The uniformity of droplet size and distribution is enhanced by this vibratory process thereby achieving greater mass transport, but the mixture is easily separated when the vibratory agitation is removed. Tuning the process between a frequency between about 15 Hz to about 1,000 Hz with an amplitude between about 0.01 mm to about 50 mm optimizes the transfer of acoustic energy into the fluid. This energy then generates an even distribution of droplets (larger than those generated with typical related processes) which collide with each other to affect mass transfer from one droplet to another. After the acoustic energy is removed, the liquids easily and quickly separate thus effecting high mass transfer without creating an emulsion.

One embodiment of the invention is to facilitate acoustic mixing of two or more pastes or suspensions. Another embodiment of the invention is to facilitate acoustic mixing of one or more pastes and one or more gases. Another embodiment of the invention is to facilitate acoustic mixing of one or more pastes with one or more solid particles. A further embodiment of the invention is to facilitate acoustic mixing of one or more pastes with one or more solid particles with one or more gases. Acoustic mixing of pastes comprising single or multiple Active Pharmaceutical Ingredients (APIs) may be to be dried before milling and then adding the micronized product into a final formulation. A distinct advantage of acoustic mixing is that viscosities from 1 cP to greater than 1,000,000 cP can be effectively mixed.

The acoustic blender may be used to create emulsions such as those described above and this apparatus can readily be connected to spray drying systems or nebulisation systems to produce spray dried particles. In one embodiment a volatile material is acoustically blended with a second material containing active material, for example a pharmaceutically active material. During the course of spray drying the volatile material migrates to the surface of the droplet containing the active material. After volatilisation of the volatile material during the spray drying process, a particle is left which has multiple dimples (resembling a golf ball) or connected holes (resembling a practice golf ball) on the surface or combinations thereof. The acoustic blender is highly efficient at minimizing the size of the volatile material, which in turn dictates the size of the holes or dimples in the final product. Volatile materials are those know to the person skilled in the art and importantly will be selected, used and treated with an abundance of caution when spray drying.

The acoustic blender may be used to create suspensions such as those described above and this apparatus can readily be connected to spray drying systems or nebulisation systems to produce spray dried particles. A volatile material is acoustically blended with a second material containing active material, for example a pharmaceutically active material. During the course of spray drying the volatile material migrates to the surface of the droplet containing the active material. After volatilisation of the volatile material during the spray drying process, a particle is left which has multiple dimples (resembling a golf ball) or connected holes (resembling a practice golf ball) on the surface or combinations thereof. The acoustic blender is highly efficient at minimizing the size of the volatile material, which in turn dictates the size of the holes or dimples in the final product. Volatile materials are those know to the person skilled in the art and importantly will be selected, used and treated with an abundance of caution when spray drying.

In one embodiment the acoustic blender may be used to create suspensions such as those described above for use in a pMDI.

In an alternate embodiment, the acoustic mixer contains a plurality of fixed deagglomerators for example a plurality of fixed sieves within the deagglomeration chamber. The sieves may have varying mesh sizes for example 63 μm, 90 μm, 125 μm, 150 μm, 212 μm etc. Most pharmaceutical powders can be sieved quickly with a standard sieve; however, some pharmaceutical powders have irregular-shaped particles or are cohesive, which can cause mesh-blinding due to problematic particles obstructing the aperture of the mesh. Screen blinding is a common problem when sieving difficult powders, typically those particles with a size of 175 μm and below. Screen blinding occurs when either one or a combination of problematic particles rest on or in an aperture of the mesh and stays there, or particles simply attach to the mesh wires occluding the aperture. When screen blinding occurs, the size of the particles falling to the next stack is then reduced. Alternatively, in the case of complete occlusion, it prevents particles from passing through these openings entirely. When screen blinding occurs, the useful screening area is reduced and, therefore, sieving capacity drops. When powders are mixed according to this embodiment, the sieves screens act as either a barrier to preclude mixing of certain particles or the screen acts to facilitate the deagglomeration and blending process.

In another embodiment unsieved lactose may be added on top of a sieve screen within the acoustic mixer. Drug particles and additive may reside below the sieve screen and the process results in a one-step sieving and blending process. The height of the screen can be manipulated to avoid any drug entering the unscreened lactose held by the screen.

In an alternate embodiment, the acoustic mixer contains a plurality of compartments with shared walls along the length of the chamber of the acoustic mixer. Each compartment is designed to hold its own formulation constituent with associated sieve screen size. For example the first compartment may contain unsieved carrier particles with its dedicated screen size, the second compartments may contain unsieved excipient particles with its dedicated screen size and a third compartments may contain unsieved drug particles with its own dedicated screen size. In an alternate embodiment, a compartment of the chamber of the acoustic mixer may contain a combination of these materials.

In an alternate embodiment, the acoustic mixer contains multiple containers with separate formulations to be mixed at the same time. This affords the convenience of avoiding cross contamination. Similarly in the event formulation components require separate conditioning, this can be achieved until the final formulation needs to be assembled.

Traditional blending approaches require the presence of layers of material of one layer (n) placed upon the other (m) to form n/m/n/m/n etc. In one embodiment, the blends produced do not require ordered layering (sandwiching) of the materials in order to achieve a homogenous blend as determined by the coefficient of variation and acceptable aerosol performance impaction analysis.

Physical reactions such as heat transfer, mass transfer and suspension of particles are greatly accelerated by exposing the reactants to a vibratory environment that is able to vibrate the reactants at a preferred frequency between about 15 Hz to about 1,000 Hz with an amplitude between about 0.01 mm to about 50 mm. By placing media containing the reactants in such an environment, the physical forces that generate these reactions are driven at higher rates. Similarly, chemical reactions are increased in rate due to enhanced contact and micromixing. The increased rate of media contact and breaking or reduction of boundary layers drives the reactions to occur at increased rates.

In one embodiment, method of the invention will, if the acoustic mixer is suitably arranged, produce composite active particles. The inhalable composite active particles are very fine particles of active material which have, upon their surfaces, an amount of the additive material. In one embodiment the additive material is in the form of a coating on the surfaces of the particles of active material. The coating may be a discontinuous coating. The additive material may be in the form of particles adhering to the surfaces of the particles of active material.

During the acoustic mixing, particles of active and additive material violently collide against each other with enough energy to locally heat and soften, break, distort, flatten and wrap the additive particles around the core active particle to form a particulate coating of additive on the active particle. The energy is generally sufficient to break up agglomerates but negligible size reduction of both components may occur. Unlike a blending or mixing process, in one embodiment, method involves high energy parameters combined within a confined space which maximises the number high energy collisions between the particles resulting in a particulate coating of additive on the active particle.

Unlike the blending or mixing process disclosed herein, in one embodiment, a method is disclosed for making composite active particles for use in a pharmaceutical composition for pulmonary administration, the method comprising acoustically milling particles of active material in the presence of particles of an additive material. This process affords sufficient energy to the particles to sufficiently break-up any agglomerates of both active material and additive material, and ensure an even distribution of the particulate additive material over the active material, and so that the particles of additive material become fused to the surface of the particles of active material, wherein the additive material may be suitable for the promotion of the dispersal of the composite active particles upon actuation of an inhaler, wherein the acoustic milling step comprises adherent particles of additive material and blending these with particles of active material.

Alternatively, composite active particles may be made by acoustically blending active material with hollow microspheres. The hollow microspheres may be those referred to in Pharmaceutical Research, Vol. 25, No. 5, May 2008. The hollow microspheres are acoustically blended with active particles that are less than 2 μm, less than 1 μm, less than 0.5 μm and less than 0.25 μm. In one embodiment, a composite particle for use in a pharmaceutical composition for pulmonary administration, the composite particle comprising a hollow porous microsphere particle enveloping an active particle, the composite particles having a mass median aerodynamic diameter of not more than 10 μm. The advantage of acoustically blending hollow porous microsphere particles with active particle is that the acoustic mixer is efficient at filling the microsphere with active but delicate enough not to destroy the structure of the hollow porous microsphere and thereby retain the benefits of these aerodynamically light particles. The vibration of active particles with the hollow microsphere in close proximity enables the fine active to engage with the holes located on the surface of the microsphere and percolate into the hollow microsphere.

Alternatively, composite active particles may be created by acoustically blending a paste containing active material with hollow microspheres. The paste permeates the hollow microspheres assisted by the acoustic blending. In one embodiment, a composite particle for use in a pharmaceutical composition for pulmonary administration, the composite particle comprising a hollow porous microsphere particle enveloping a paste or suspension, the composite particles having a mass median aerodynamic diameter of not more than 10 μm.

Alternatively, composite active particles may be made by acoustically blending active material with multiple additives. In one embodiment, the composite active particles are created by sequentially adding an additive to the blend until a uniform coating of the active particles is achieved. In one embodiment, a composite particle for use in a pharmaceutical composition for pulmonary administration, the composite particle comprising an active particle enveloped with layers of additive particle, the composite particles having a mass median aerodynamic diameter of not more than 10 μm and wherein the layers are 1 layer of additive, at least 1 layer of additive, 2 layers of additive, 3 layers of additive, or at least 3 layers of additive on the active particle.

This intensive process creates composite active particles for use in a pharmaceutical composition for pulmonary administration, each composite active particle comprising a particle of active material and a particle of additive material on the surface of that particle of active material, wherein the composite active particles have a mass median aerodynamic diameter of not more than 15 μm, not more than 10 μm, not more than 7 μm or not more than 5 μm and wherein the additive material promotes the dispersion of the composite active particles upon actuation of a delivery device. In one embodiment the additive particle is softer than the active particle.

In one embodiment the additive particle is of equivalent size to the active particle. Alternatively, the additive particle is of a smaller size than the active particle or alternatively, the additive particle is of a larger size than the active particle. The sizes referred to above may be mass median aerodynamic diameters.

Alternatively, composite active particle made using intensive milling techniques may be added to the acoustic mixer for assembly into a final blend. Suitable milling methods are those involving the Mechano-Fusion, Hybridiser and Cyclomix instruments. In one embodiment, the milling step involves the compression of the mixture of active and additive particles in a gap (or nip) of fixed, predetermined width (for example, as disclosed in WO 2002/43701).

Alternatively, composite excipient particle made using intensive milling techniques may be added to the acoustic mixer for assembly into a final blend. Suitable milling methods are those involving the Mechano-Fusion, Hybridiser and Cyclomix instruments. In one embodiment, the milling step involves the compression of the mixture of excipient and additive particles in a gap (or nip) of fixed, predetermined width (for example, as disclosed in WO 2002/000197).

Low shear mixing applications are necessary to prevent or reduce damage to pharmaceutical formulations. This is achieved by placing the pharmaceutical formulations in a vibratory environment that is operative to vibrate the pharmaceutical formulations at a frequency of about 5 Hz to about 1,000 Hz with an amplitude between about 0.01 mm to about 50 mm. The pharmaceutical formulations are physically mixed with gases, solids and liquids in an environment of low shear and minimal particle to particle collisions. Particles are prevented from agglomerating into large agglomerates.

In one embodiment, the acoustic mixer contains dampeners within the formulation. These dampeners are designed to modify and absorb the energy entering the formulation thereby avoiding damaging delicate particles within the formulation, for example fused granulated carrier particles. These dampeners may be balloons, hollow balls, light polystyrene particles or any similar particle. These dampeners may be recovered from the formulation by sieving when required.

Intrusion or infusion of gases entrained into a solid media is enhanced by placing the solid media in an environment that is operative to vibrate the solid media at a frequency of about 5 Hz to about 1,000 Hz with an amplitude between 0.01 mm to about 50 mm. Boundary layers are broken and gases are forced into, out of and through the particulate structure.

Another embodiment of the invention is to cause vapour to permeate through the fluidised powder bed. In one embodiment, the acoustic mixer is connected to a conditioning apparatus, for blending and conditioning the formulation (or constituents thereof prior to assembling the formulation). In one aspect, the active ingredient may be conditioned under conditions of low relative humidity whilst the acoustic mixer is in operation. In one embodiment, the active is treated under conditions of less than 10% relative humidity whilst the acoustic mixer is in operation. In one embodiment, the active is treated under conditions of between 0.5% and 10% relative humidity, in one embodiment between 2% and 9%, in one embodiment between 3% and 8%, in one embodiment between 4% and 7%, in one embodiment between 4% and 6%, or in one embodiment less than 5%, whilst the acoustic mixer is in operation.

In one aspect, a method is disclosed, for making a pharmaceutical composition, the method comprising a step in which an inhalable pharmaceutically active material is acoustically blended by exposure to reduced level of relative humidity as compared to ambient conditions, wherein the acoustic frequency operating range is from 5 Hz to about 1,000 Hz for a period of at least 2 minutes. Acceptable conditioning may be determined by a sustained D₉₀≦20 μm for more than 1 week, preferably more than 1 month, preferably more than 3 months or more preferably more than 9 months.

In one aspect, the active ingredient may be conditioned under a humid atmosphere whilst the acoustic mixer is in operation. In one embodiment, the active ingredient is conditioned under a relative humidity ranging from 5 to 90%. When intending to process under conditions of higher humidity, relative humidity ranges from 50 to 90%, 55 to 87%, 60 to 84%, 60 to 80%, 65 to 80%, 70 to 75% or 70 to 80% are preferred. In one aspect, the active ingredient may be conditioned under conditions of higher humidity, relative humidity that ranges from 51 to 100%, 61 to 100%, 71 to 100%, 81 to 100% or 91 to 100% are suitable embodiments. When intending to process under conditions of reduced humidity, ranges are from 5 to 50%, 7.5 to 40%, 10 to 30%, 12.5 to 20% and in one embodiment less than 15% relative humidity are suitable. In the case of cryogenic preparation, for example with the use of liquid nitrogen, reduced humidity ranges will be less than 5%.

In one aspect, a method is disclosed, for making a pharmaceutical composition, the method comprising a step in which an inhalable pharmaceutically active material is acoustically blended by exposure to elevated level of relative humidity as compared to ambient conditions, wherein the acoustic frequency operating range is from 5 Hz to about 1,000 Hz for a period of at least 2 minutes. Acceptable conditioning may be determined by a sustained D₉₀≦20 μm for more than 1 week, preferably more than 1 month, preferably more than 3 months or more preferably more than 9 months.

In one aspect, the active ingredient may be conditioned under a solvent containing atmosphere, such as an organic solvent whilst the acoustic mixer is in operation. Solvents include alcohols and/or acetone. The skilled artisan would appreciate the nature of risk associated with processing under such environments. Suitable environments include ethanol/nitrogen in ratios of 5:95% (w/w), in one embodiment 2.5:97.5% (w/w) in one embodiment 1:99% (w/w). Alternatively methanol/nitrogen in ratios of 5:95% (w/w), in one embodiment 2.5:97.5% (w/w) in one embodiment 1:99% (w/w) may be used. Alternatively acetone/nitrogen in ratios of 5:95% (w/w), in one embodiment 2.5:97.5% (w/w) in one embodiment 1:99% (w/w) may be used. The solvent may be introduced as a vapour within the gas lines to the acoustic mixer. The solvent may be introduced as a vapour in increasing amounts, from ambient for a length of time, for example, then increasing or decreasing by not more than 5% (w/w), not more than 10% (w/w), not more than 15% (w/w), not more than 20% (w/w) or alternatively not more than 25% (w/w) from the initial baseline and then optionally returning the vapour amount to baseline whilst the acoustic mixer is in operation. Alternatively, the solvent may be introduced as a vapour in increasing amounts, from 0% for a length of time, for example, then increasing by 1% (w/w) increments whilst the acoustic mixer is in operation until a desired vapour concentration is achieved. Alternatively, once a steady vapour state is achieved the solvent vapour may be decreased within the vessel with processing time, either during operation of the acoustic mixer or afterwards. Humidity may also be varied over time during the treatment of the active ingredient. The length of time to which the particles are exposed to this humidity may also be varied.

When used herein, “water” is neither an excipient nor an additive material.

Conditioning of the formulation or its constituent parts may take place before, during and/or after operating the acoustic mixer.

In another aspect the acoustic mixing may take place in a vacuum. In another aspect the acoustic mixing may take place under a pressurised environment.

Another embodiment of the invention is to accelerate physical and chemical reactions. A further embodiment of the invention is to accelerate heat transfer away from heat-sensitive materials. Another embodiment of the invention is to accelerate mass transfer. Yet another embodiment of the invention is to suspend and distribute particles. A further embodiment of the invention is to distribute particles. Another embodiment of the invention is to cause micromixing.

In another aspect the active ingredient is conditioned at a minimum temperature whilst the acoustic mixer is in operation. In one embodiment, the temperature is at least 30° C., in one aspect 35° C., in one aspect 40° C., in one aspect 50° C., or higher than 50° C. Processing temperatures may be controlled via an external or integrated cooling jacket. Alternatively, the processing temperature may also be controlled via a suitably heated or cooled atmosphere. Alternatively, temperature may also be varied over time during the treatment of the active ingredient. For example the heated atmosphere may be introduced by increasing temperature with processing time until the desired temperature is achieved. Alternatively, once a steady heated state is achieved the temperature may be decreased within the vessel with processing time.

A particular advantage of blending with an acoustic mixer is that a minimal rise in temperature following formulation processing is obtained, even after extended processing periods. In one embodiment, the temperature rise following blending is no more than 5° C., in one aspect no more than 10° C., in one aspect no more than 15° C., in one aspect no more than 20° C., or in one aspect no more than 30° C. In each of these aspects blend completion is determined by a CV of less than 5%. In one embodiment, use of an additive material in a pharmaceutical composition for pulmonary administration, wherein the additive material is suitable for minimising an increase in blend temperature during blending as compared with the same blend and process in the absence of the additive material. Suitable additive materials for this purpose include magnesium stearate.

In order to determine the initial homogeneity, the quantity of drug (as determined by, for example, HPLC) in each sample is expressed as a percentage of the original recorded weight of the powder sample. The values for all the samples are then averaged to produce a mean value, and the coefficient of variation (CV) around this mean is calculated. The coefficient of variation is a direct measure of the homogeneity of the mix. A powder, whose homogeneity measured as a percentage coefficient of variation, is less than about 5% can be regarded as acceptable and a coefficient of variation of 2% is excellent.

In one aspect the additive material is an anti-adherent material that will tend to decrease the cohesion between the active ingredient, and between the active ingredient and other particles present in the pharmaceutical composition.

The additive material may be an anti-friction agent (glidant), suitably to give better flow of the pharmaceutical composition in, for example, a dry powder inhaler which will lead to a better dose reproducibility.

Where reference is made to an anti-adherent material, or to an anti-friction agent, the reference is to include those materials which are able to decrease the cohesion between the particles, or which will tend to improve the flow of powder in an inhaler, even though they may not usually be referred to as anti-adherent material or an anti-friction agent. For example, leucine is an anti-adherent material as herein defined and is generally thought of as an anti-adherent material but lecithin is also an anti-adherent material as herein defined, even though it is not generally thought of as being anti-adherent, because it will tend to decrease the cohesion between the active ingredients and between the active ingredient and other particles present in the pharmaceutical composition.

The additive material may be in the form of particles which tend to adhere to the surfaces of active ingredient, as disclosed in WO1997/03649. Alternatively, the additive material may be coated on the surface of the active ingredient by a co-milling method, as disclosed in WO2002/43701. Therefore, in one aspect of the invention, the method may further comprise and additional step of coating the surface of the active ingredient with an additive material (e.g. by a co-milling method).

The additive material may include one or more compounds selected from amino acids and derivatives thereof, and peptides and derivatives thereof. Amino acids, peptides and derivatives of peptides are suitably physiologically acceptable and give acceptable release of the active ingredient on inhalation.

The additive may comprise one or more of any of the following amino acids: leucine, isoleucine, lysine, valine, methionine, and phenylalanine. The additive may be a salt or a derivative of an amino acid, for example aspartame or acesulfame K. Alternatively, the additive consists substantially of an amino acid, or of leucine, advantageously L-leucine. The L-, D and DL-forms of an amino acid may also be used. As indicated above, leucine has been found to give particularly efficient dispersal of the active ingredient on inhalation.

The additive may include one or more water soluble substances. A water soluble substance may be a substance that may be capable of dissolving wholly or partly in water and which is not entirely insoluble in water. This may help absorption of the additive by the body if it reaches the lower lung. The additive may include dipolar ions, which may be zwitterions. It is also advantageous to include a spreading agent as an additive, to assist with the dispersal of the composition in the lungs. Suitable spreading agents include surfactants such as known lung surfactants (e.g. ALEC™) which comprise phospholipids, for example, mixtures of DPPC (dipalmitoyl phosphatidylcholine) and PG (phosphatidylglycerol). Other suitable surfactants include, for example, dipalmitoyl phosphatidyl than olamine (DPPE), dipalmitoyl phosphatidylinositol (DPPI).

The additive may comprise a metal stearate, or a derivative thereof, for example, sodium stearyl fumarate or sodium stearyl lactylate. Advantageously, it comprises a metal stearate, for example, zinc stearate, magnesium stearate, calcium stearate, sodium stearate or lithium stearate. In one embodiment, the additive material comprises magnesium stearate, for example vegetable magnesium stearate, or any form of commercially available metal stearate, which may be of vegetable or animal origin and may also contain other fatty acid components such as palmitates or oleates.

The additive may include or consist of one or more surface active materials. A surface active material may be a substance capable reducing the surface tension of a liquid in which it is dissolved. Surface active materials may in particular be materials that are surface active in the solid state, which may be water soluble or water dispersible, for example lecithin, in particular soya lecithin, or substantially water insoluble, for example solid state fatty acids such as oleic acid, lauric acid, palmitic acid, stearic acid, erucic acid, behenic acid, or derivatives (such as esters and salts) thereof such as glyceryl behenate. Specific examples of such materials are phosphatidylcholines, phosphatidylethanolamines, phosphatidylglycerols and other examples of natural and synthetic lung surfactants; lauric acid and its salts, for example, sodium lauryl sulphate, magnesium lauryl sulphate; triglycerides such as Dynsan 118 and Cutina HR; and sugar esters in general. Alternatively, the additive may be cholesterol.

Other possible additive materials include sodium benzoate, hydrogenated oils which are solid at room temperature, talc, titanium dioxide, aluminium dioxide, silicon dioxide and starch. Also useful as additives are film-forming agents, fatty acids and their derivatives, as well as lipids and lipid-like materials.

In one aspect, additive particles are composed of lactose. The additive particles may be lactose fines. The additive lactose may be added a various stages of the formulation assembly or the additive lactose may be formed as a result of processing of a larger lactose carrier particle. Said processing cleaves off the protruding asperities and produces smaller lactose particles that may re-adhere to the larger carrier particles or combine with different components of the composition.

A particular advantage of magnesium stearate in acoustic powder blending is it minimises a rise in formulation temperature during processing with an acoustic mixer as determined by experimentation in the absence of magnesium stearate. The presence of magnesium stearate in the blend also maintains acceptable blend homogeneity as determined by the coefficient of variation and acceptable aerosol performance as determined by aerosol impaction analysis. After completion of the blending, the blending times were extended still further and even after these extended processing periods negligible rises in formulation temperature were observed. In one aspect, additive particles comprise magnesium stearate.

In one aspect a plurality of different additive materials can be used. In one embodiment combinations of additive materials include lactose fines and magnesium stearate. In one embodiment the lactose fines and magnesium stearate are in loose association. Alternatively, the magnesium stearate is smeared or fused over the particles of fine lactose.

Carrier particles may be of any acceptable inert excipient material or combination of materials. For example, carrier particles frequently used in the prior art may be composed of one or more materials selected from sugar alcohols, polyols and crystalline sugars. Other suitable carriers include inorganic salts such as sodium chloride and calcium carbonate, organic salts such as sodium lactate and other organic compounds such as polysaccharides and oligosaccharides. Advantageously, the carrier particles comprise a polyol. In particular, the carrier particles may be particles of crystalline sugar, for example mannitol, dextrose or lactose. In one embodiment, the carrier particles are composed of lactose. Suitable examples of such excipient include LactoHale 300 (Friesland Foods Domo), LactoHale 200 (Friesland Foods Domo), LactoHale 100 (Friesland Foods Domo), PrismaLac 40 (Meggle), InhaLac 70 (Meggle).

Alternatively, composite carrier particles may be made by acoustically blending carrier material with additive. In one embodiment, the composite carrier particles are created by sequentially adding an additive to the blend until a coating of the carrier particles is achieved. In one embodiment, a composite carrier particle for use in a pharmaceutical composition for pulmonary administration, the composite particle comprising a carrier particle enveloped with a layer of additive particle, the composite particles having a diameter of greater than 63 μm and wherein the layers are 1 layer of additive, at least 1 layer of additive, 2 layers of additive, 3 layers of additive, or at least 3 layers of additive on the carrier particle. A composition comprising particles falling within the scope of this embodiment will easily recover these particles via a 63 μm sieve screen.

Alternatively, composite carrier particles may be made by acoustically blending carrier material with active. In one embodiment, the composite carrier particles are created by sequentially adding an active to the blend until a coating of the carrier particles is achieved. In one embodiment, a composite carrier particle for use in a pharmaceutical composition for pulmonary administration, the composite particle comprising a carrier particle enveloped with a layer of additive particle, the composite particles having a diameter of greater than 63 μm and wherein the layers are 1 layer of active, at least 1 layer of active, 2 layers of active, 3 layers of active, or at least 3 layers of active on the carrier particle. A composition comprising particles falling within the scope of this embodiment will easily recover these particles via a 63 μm sieve screen. In one embodiment, the layers may comprise alternate layers of active. For example, additive 1 coated by additive 2 which is in turn coated by additive 1.

The ratio in which the carrier particles (if present) and active ingredient are mixed will depend on the type of inhaler device used, the type of active particle used and the required dose. The carrier particles may be present in an amount of at least 50%, at least 70%, at least 90% and at least 95% based on the combined weight of the active ingredient and the carrier particles and additives, if additive is present.

Wet granulation is a process in which a mix of powders is agglomerated with a liquid binder forming larger particles or granules. These granules normally have a size distribution in the range of 100 μm to 2000 μm, and are mainly used for tablet compaction and capsule filling. Wet granulation is typically used to improve the flow, compressibility and homogeneity of the mixture used to produce solid dosage forms. The most widely used excipients for granulation are microcrystalline cellulose, lactose and dibasic calcium phosphate. The three main types of wet granulation process are (i) low shear granulation using a planetary mixer, (ii) high shear granulation using a high speed mixer with an impeller and chopper and (iii) fluid-bed granulation using fluid-bed drier.

These granulated lactose particles are particularly useful in inhalable formulations because they have a multitude of clefts and crevices in which the drug particles may reside. However, they require delicate blending approaches to avoid damaging their fragile structures. This has meant that until now, these shear sensitive granulated lactose particles required prolonged blending times at lower energy levels to maintain their physical structure. Surprisingly we have found that blends comprising granulated lactose particles can be achieved in much shorter periods of times whilst still possessing acceptable blend homogeneity as determined by the coefficient of variation, acceptable aerosol performance as determined by aerosol impaction analysis and still maintain their physical size as determined by microscopy and particle size analysis.

In one embodiment the use of an acoustic blender for the preparation of a pharmaceutical composition wherein the pharmaceutical composition possesses at least equivalent or better blend homogeneity, at least equivalent or better aerosol performance as compared with the same starting formulation processed by a TRV blender but wherein the blend homogeneity is obtained in less than 90%, less than 80%, less than 70%, less than 60%, less than 50%, less than 40%, less than 30%, less than 20% or less than 10% of the blend time taken by the TRV blender, wherein the composition is an inhalable composition for treatment of respiratory diseases.

In one embodiment the use of an acoustic blender for the preparation of a pharmaceutical composition wherein the pharmaceutical composition possesses at least equivalent or better blend homogeneity, at least equivalent or better aerosol performance as compared with the same starting formulation processed by a Diosna but wherein the blend homogeneity is obtained in less than 90%, less than 80%, less than 70%, less than 60%, less than 50%, less than 40%, less than 30%, less than 20% or less than 10% of the blend time taken by the Diosna, wherein the composition is an inhalable composition for treatment of respiratory diseases.

Alternatively, composite carrier particles may be made by acoustically blending active material onto the carrier particles. In one embodiment, alternate layers of a first active material followed by a second active material followed by the first active material may be used to coat the carrier particles.

In one embodiment, composite carrier particles are created by sequentially adding an additive to the blend until a uniform coating of the carrier particles is achieved. In one embodiment, a composite particle for use in a pharmaceutical composition for pulmonary administration, the composite particle comprising an carrier particle enveloped with layers of additive particle, the composite carrier particles having a diameter of more than 50 μm and wherein the layers are 1 layer of additive, in one embodiment at least 1 layer of additive, in one embodiment 2 layers of additive, in one embodiment 3 layers of additive, or in one embodiment at least 3 layers of additive on an active particle.

An alternative embodiment provides an active ingredient for use in a pharmaceutical composition, a pharmaceutical composition for inhalation, in one embodiment a powder for a dry powder inhaler. In one embodiment, the active ingredient may be for use in a pharmaceutical composition for a pressurized metered dose inhaler (pMDI).

In another embodiment of the present invention, powders in accordance with the present invention may be administered using active or passive devices. In one embodiment of the invention, the inhaler device is an active device, in which a source of compressed gas or alternative energy source is used. Examples of suitable active devices include Aspirair™ (Vectura), Microdose™ and the active inhaler device produced by Nektar Therapeutics (as covered by U.S. Pat. No. 6,257,233).

In an alternative embodiment, the inhaler device is a passive device, in which the patient's breath is the only source of gas which provides a motive force in the device. Examples of “passive” dry powder inhaler devices include the Rotahaler™ and Diskhaler™ (GlaxoSmithKline) and the Turbohaler™ (AstraZeneca), Monohaler™ (Miat), GyroHaler™ (Vectura) and Novolizer™ (Viatris GmbH).

The size of the doses can vary from micrograms to milligrams, depending upon the active ingredient, the delivery device and disease to be treated. Suitably the dose will range from 1 ng to 50 mg of active ingredient, in one embodiment 10 mg to 20 mg and in one embodiment 100 μg to 10 mg. The skilled artisan will appreciate that dose of the active will depend on the nature of the active pharmaceutical ingredient, therefore a dose of 1 mg to 10 mg, in one embodiment 2 mg to 8 mg, in one embodiment 3 mg to 7 mg and in one embodiment 4 mg to 5 mg is required. Alternatively a dose of 5 mg to 15 mg, a dose of 6 mg to 14 mg, in one embodiment 7 mg to 13 mg and in one embodiment 8 mg to 12 mg is required. Alternatively a dose of 10 mg to 20 mg, in one embodiment 12 mg to 18 mg, in one embodiment 14 mg to 16 mg and in one embodiment 14.5 mg to 15.5 mg is required. Alternatively a dose of 20 mg to 25 mg, more preferably in one embodiment 2 1 mg to 24 mg, in one embodiment 22 mg to 23 mg and in one embodiment 22.5 mg is required. Doses referred to above are nominal doses. These amounts should not be confused with the total amount of the pharmaceutical composition that is prepared.

Reference to doses herein is generally a reference to metered doses (MD) (or nominal doses (ND), the two terms may be used interchangeably). The MD is the dose of active pharmaceutical ingredient in the blister or capsule or formulation holding receptacle.

The emitted dose (ED) or delivered dose (DD) (the two terms may be used interchangeably) is the total mass of the active agent emitted from the device following actuation. It does not include the material left on the internal or external surfaces of the device, or in the metering system including, for example, the capsule or blister. The ED is measured by collecting the total emitted mass from the device in an apparatus frequently identified as a dose uniformity sampling apparatus (DUSA), and recovering this by a validated quantitative wet chemical assay (a gravimetric method is possible, but this is less precise).

The fine particle dose (FPD) is the total mass of active agent which is emitted from the device following actuation which is present in an aerodynamic particle size smaller than a defined limit. This limit is generally taken to be 5 μm MMAD if not expressly stated to be an alternative limit, such as 3 μm, 2 μm or 1 μm, etc.

The fine particle fraction (FPF) is normally defined as the FPD (the dose that is <5 μm MMAD) divided by the delivered Dose (DD) which is the dose that leaves the device. The FPF is expressed as a percentage. Herein, the FPF of DD is referred to as FPF (DD) and is calculated as FPF (DD)=(FPD/DD)×100%.

The fine particle fraction (FPF) may also be defined as the FPD divided by the Metered Dose (MD) which is the dose in the blister or capsule, and expressed as a percentage. Herein, the FPF of MD is referred to as FPF (MD), and may be calculated as FPF (MD)=(FPD/MD)×100%.

According to an embodiment of the present invention, a receptacle is provided, holding a dose of the active ingredient prepared according to the present invention. The receptacle may be a capsule or blister, or a foil blister.

Active ingredient, suitably in the form of a powder, in accordance with the present invention may be pre-metered. The powders may be kept in foil blisters which offer chemical and physical protection whilst not being detrimental to the overall performance. Indeed, the formulations thus packaged tend to be stable over long periods of time, which is very beneficial, especially from a commercial and economic point of view.

In one embodiment, the composition according to the present invention is held in a receptacle containing a single dose of the powder, the contents of which may be dispensed using one of the aforementioned devices. Reservoir devices may also be used.

The invention also relates to a method of acoustically processing an active ingredient, the method comprising submitting an active ingredient to vibrational processing in the absence of another powder material, optionally then combining the active ingredient with another agent, such as another active ingredient, an excipient or additive, and then packaging the active ingredient into a receptacle or drug delivery device.

The ingredient may be combined with other components of a pharmaceutical composition, such as an active ingredient or excipient. In one aspect such other components may also have been subjected to compression and shearing forces in the absence of another powder material.

In one embodiment of the present invention there is provided a composition, in one embodiment a pharmaceutical composition, comprising an active ingredient made by a method according to the present invention in combination with an additional ingredient such as an additive, carrier and/or flavouring agent or other excipient.

The use of an acoustic mixer in the context of a formulation blend confers a number of distinct advantages. Firstly, the absence of agitators blades or impellers in the mixing chamber minimizes and destruction of delicate structures within the blend. Unlike the localised mixing produced by blades and impellors an acoustic mixer provides a uniform shear field throughout the mixing chamber. The use of an acoustic mixer avoids “dead zones” in the mixing chamber where efficient mixing does not take place. This is particularly useful when attempting to obtain uniform blends. The acoustic mixing chamber can be used as the shipping container. This affords the benefit of conducting the mixing process in one location and shipping the entire blend to a completely new location, where, for example, a powder filling line may be located in a different country. This benefit is particularly useful when a blend must be filled into capsules or blisters because intermittent agitation may be employed during the blister/capsule filling process. This intermittent agitation avoids problems such as blocking of the hopper caused by “rat holes” within the powder blend. The term “rat hole” describes the phenomenon wherein powder particles form temporary bridges thereby holding the formulation above the bridge in place whilst the formulation below the bridge collapses creating a formulation cavity. One of the key technical challenges in manufacturing a powder blend is the inability to migrate from laboratory bench scale through to commercial scale. This obstacle is encountered because the particle physics relevant to laboratory bench scale do not translate to a commercial scale arrangement. Due to the advantage of a uniform shear field throughout the mixing chamber irrespective of the scale use, the scale up procedures are more straightforward. Finally, the most distinct benefit of the acoustic mixer is the benefit of shorter blending times compared with traditional Turbula or Tumble mixers.

In one embodiment, a method is disclosed for making a pharmaceutical composition, the method comprising a step in which an inhalable pharmaceutically active material is acoustically blended with excipient material, wherein the acoustic frequency operating range is from 5 Hz to about 1,000 Hz for a period of at least 2 minutes until a coefficient of variation of less than 5% is achieved and wherein the acoustically blending vessel containing the blended pharmaceutical composition may then attach to an automated filling apparatus. Preferably, wherein the excipient material comprises lactose.

Drugs which may be used in formulations to be process by acoustic mixing include the following:

Bronchodilators (e.g. the β2-agonists bambuterol, bitolterol, broxaterol, carmoterol, clenbuterol, fenoterol, formoterol, indacaterol, levalbuterol, metaproterenol, orciprenaline, picumeterol, pirbuterol, procaterol, reproterol, rimiterol, salbutamol, salmeterol, terbutaline, vilanterol and the like);

Anti-muscarinics (e.g. ipratropium, ipratropium, bromide, oxitropium, tiotropium and glycopyrrolate);

Antibiotic and antibacterial agents (e.g. including the beta-lactams, fluoroquinolones, ketolides, macrolides, sulphonamides and tetracyclines, aclarubicin, amoxicillin, amphotericin, azithromycin, aztreonam chlorhexidine, clarithromycin, clindamycin, colistimethate, dactinomycin, dirithromycin, doripenem, erythromycin, fusafungine, gentamycin, metronidazole, mupirocin, natamycin, neomycin, nystatin, oleandomycin, pentamidine, pimaricin, probenecid, roxithromycin, sulphadiazine and triclosan);

Anti-infective agents (e.g. antivirals (including nucleoside and non-nucleoside reverse transcriptase inhibitors and protease inhibitors) including aciclovir, adefovir, amantadine, cidofovir, efavirenz, famiciclovir, foscarnet, ganciclovir, idoxuridine, indinavir, inosine pranobex, lamivudine, nelfinavir, nevirapine, oseltamivir, palivizumab, penciclovir, pleconaril, ribavirin, rimantadine, ritonavir, ruprintrivir, saquinavir, stavudine, valaciclovir, zalcitabine, zanamivir, zidovudine and interferons);

aminoglycosides (e.g. tobramycin; antifungals for example amphotericin, caspofungin, clotrimazole, econazole nitrate, fluconazole, itraconazole, ketoconazole, miconazole, nystatin, terbinafine and voriconazole; antituberculosis agents for example capreomycin, ciprofloxacin, ethambutol, meropenem, piperacillin, rifampicin and vancomycin; beta-lactams including cefazolin, cefmetazole, cefoperazone, cefoxitin, cephacetrile, cephalexin, cephaloglycin and cephaloridine; cephalosporins, including cephalosporin C and cephalothin; cephamycins such as cephamycin A, cephamycin B, cephamycin C, cephapirin and cephradine);

Leprostatics (e.g. clofazimine; penicillins including amoxicillin, ampicillin, amylpenicillin, azidocillin, benzylpenicillin, carbenicillin, carfecillin, carindacillin, clometocillin, cloxacillin, cyclacillin, dicloxacillin, diphenicillin, heptylpenicillin, hetacillin, metampicillin, methicillin, nafcillin, 2-pentenylpenicillin, penicillin N, penicillin O, penicillin S and penicillin V; quinolones including ciprofloxacin, clinafloxacin, difloxacin, grepafloxacin, norfloxacin, ofloxacine and temafloxacin); tetracyclines including doxycycline and oxytetracycline;

miscellaneous anti-infectives for example linezolide, trimethoprim and sulfamethoxazole.
Nonsteroidal anti-inflammatory agents (e.g. aceclofenac, acetaminophen, alminoprofen, amfenac, aminopropylon, amixetrine, aspirin, benoxaprofen, bromfenac, bufexamac, carprofen, celecoxib, choline, cinchophen, cinmetacin, clometacin, clopriac, diclofenac, diclofenac sodium, diflunisal, ethenzamide, etodolac, etoricoxib, fenoprofen, flurbiprofen, ibuprofen, indomethacin, indoprofen, ketoprofen, ketorolac, loxoprofen, mazipredone, meclofenamate, mefenamic acid, meloxicam, nabumetone, naproxen, nimesulide, parecoxib, phenylbutazone, piroxicam, pirprofen, rofecoxib, salicylate, sulindac, tiaprofenic acid, tolfenamate, tolmetin and valdecoxib);
Other anti-inflammatory agents (e.g. B-cell inhibitors, p38 MAP kinase inhibitors, particularly, ADS115398 and TNF inhibitors);
PDE4 inhibitors (e.g. cilomilast, etazolate, rolipram, oglemilast, roflumilast, ONO 6126, tolafentrine and zardaverine); quinazolinediones (e.g. nitraquazone and nitraquazone analogs; xanthine derivatives such as denbufylline and arofylline; tetrahydropyrimidones such as atizoram; and oxime carbamates such as filaminast);
Steroids (e.g. alcometasone, beclomethasone, beclomethasone dipropionate, betamethasone, budesonide, butixocort, ciclesonide, clobetasol, deflazacort, diflucortolone, desoxymethasone, dexamethasone, fludrocortisone, flunisolide, fluocinolone, fluometholone, fluticasone, fluticasone proprionate, hydrocortisone, methylprednisolone, mometasone, nandrolone decanoate, neomycin sulphate, prednisolone, rimexolone, rofleponide, triamcinolone and triamcinolone acetonide);
Matrix metalloprotease inhibitors (e.g. adamalysins, serralysins, and astacins);
Epithelial sodium channel (ENaC) inhibitors (e.g. P-680 and Denufosol)
CFTR Potentiators (e.g. for example VX-809);
Methylxanthines (e.g. caffeine, theobromine and theophylline);
Drugs for cystic fibrosis management (e.g. Pseudomonas aeruginosa infection vaccines (eg Aerugen™), alpha 1-antitripsin, amikacin, cefadroxil, denufosol, duramycin, glutathione, mannitol, and tobramycin).

The invention further relates to an active ingredient obtainable or obtained using the above method.

The invention further relates to an inhaler device comprising an active ingredient obtainable or obtained by the method of the invention, or an active ingredient which has been further processed where necessary into a pharmaceutically acceptable form.

The invention further relates to a receptacle, such as a blister or capsule, comprising a dose of an active ingredient, obtainable or obtained by the method of the invention, or an active ingredient which has been further processed where necessary into a pharmaceutically acceptable form.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine study, numerous equivalents to the specific procedures described herein. Such equivalents are considered to be within the scope of this invention and are covered by the claims. All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the measurement, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

While certain embodiments of the present invention are described in detail above, the scope of the invention is not to be considered limited by such disclosure, and modifications are possible without departing from the spirit of the invention as evidenced by the examples and claims.

The present invention is illustrated by the by the experimental data set out below, which is not limiting upon the invention.

### BRIEF DESCRIPTION OF THE DRAWINGS

FIG. 1 shows an increase in temperature of the blend of 100 g of LH200 with 500 mg magenta toner. See Example 3.

FIG. 2 shows the relationship between mixing intensity and time to achieve blend homogeneity. See Example 4

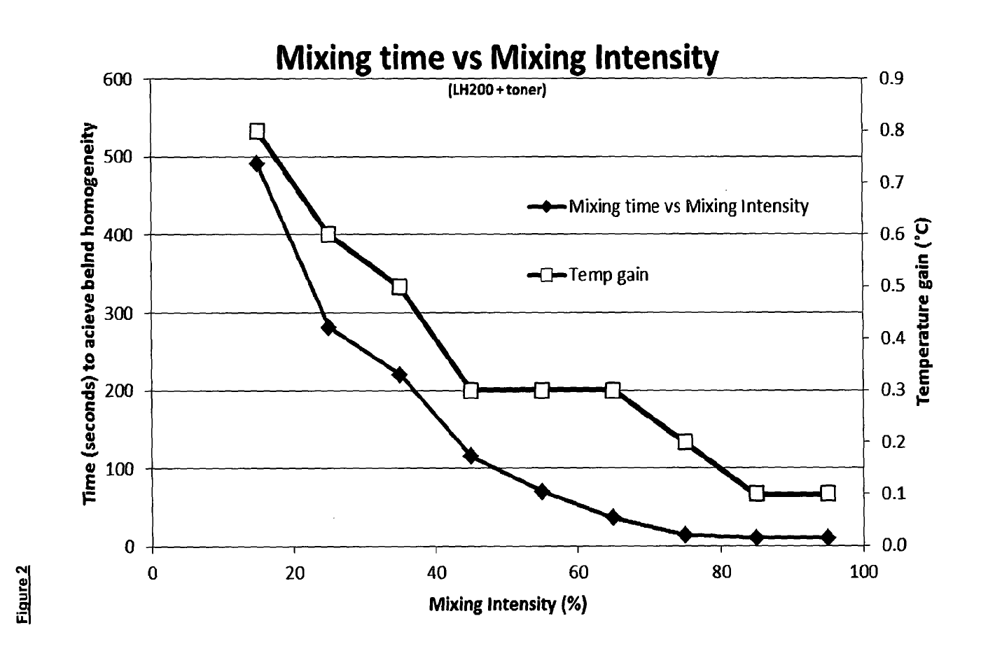

FIG. 3 shows a volume distribution of a Prismalac 355-600 blend after processing with a Diosna. D₁₀ 85.63 μm, D₅₀ 153.7 μm and D₉₀ 216.2 μm. See Table 7.

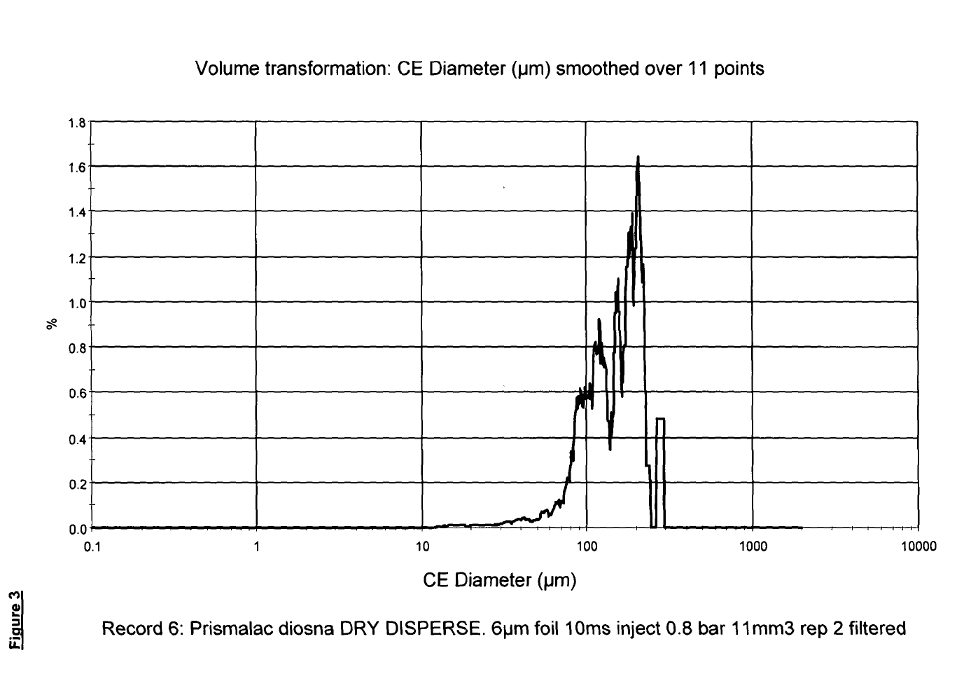

FIG. 4 shows a volume distribution of a Prismalac 355-600 blend before processing. D₁₀ 454.2 μm, D₅₀ 570.7 μm and D₉₀ 684 μm.

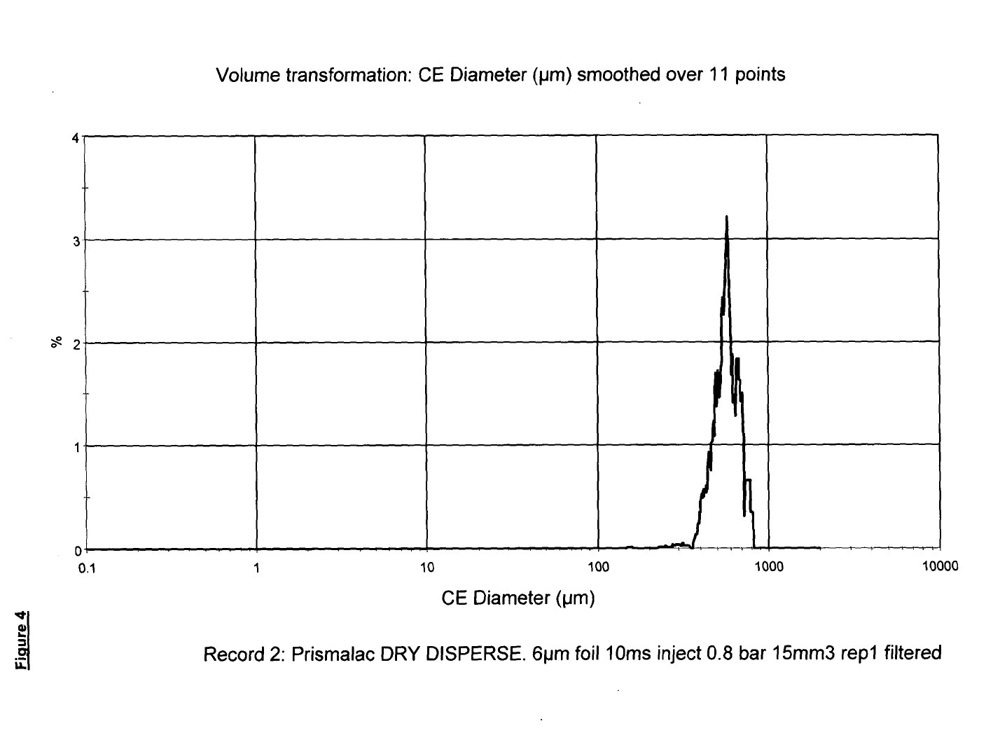

FIG. 5 shows a volume distribution of a Prismalac 355-600 blend after processing with a LabRAM. D₁₀ 500.6 μm, D₅₀ 551.1 μm and D₉₀ 776.0 μm.

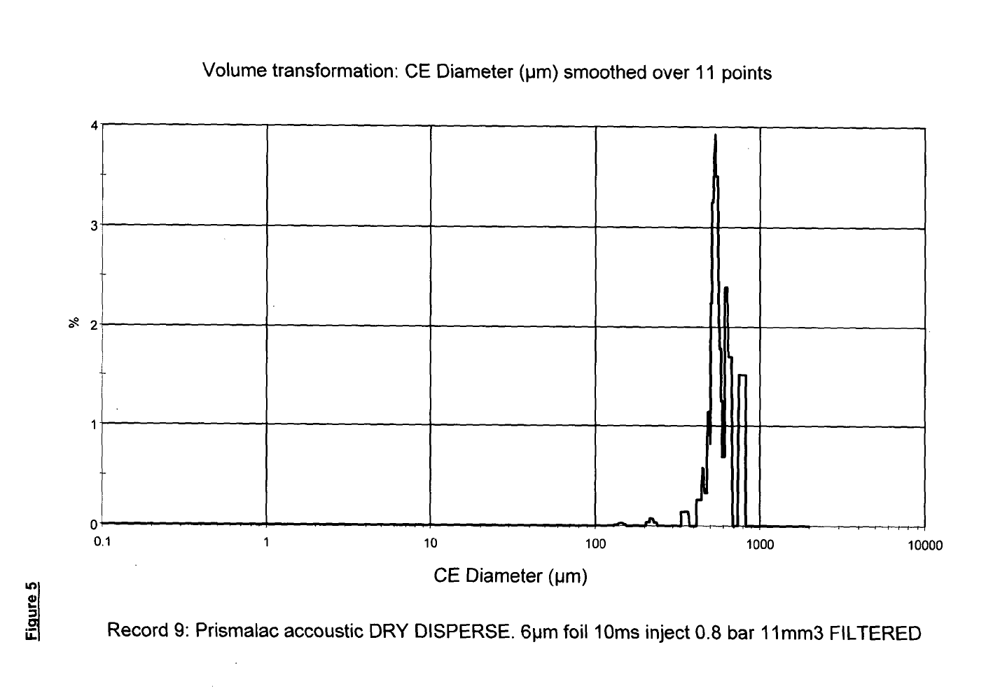

FIG. 6 shows a Prismalac 355-600 particle following LabRAM processing showing the intact connected structure of the lactose crystals.

FIG. 7 shows a powder conditioning cell that incorporates an outlet which may also incorporate a filter to ensure no emission of micronised API into the atmosphere.

### EXAMPLES

Selected embodiments of the present invention will now be explained with reference to the examples and drawings. It will be apparent to those skilled in the art from this disclosure that the following descriptions of the embodiments are for illustration only and not for the purpose of limiting the invention as defined by the appended claims and their equivalents.

### Example 1

Various grades and sieve fractions of lactose were tumble mixed using a WAB Turbula with a portion of magenta toner (Hewlett Packard, extracted from Laser Print cartridge) to allow a visual evaluation of the mixing capability.
1.1. Sorbalac 400 (100 g) was mixed with 500 mg of magenta toner in a turbula for 2 minutes at 30 rpm. The formulation did not mix as determined by visual inspection. There remained distinct regions of magenta, white and various shades of pink in the formulation.
1.2. Sorbalac 400 (100 g) was mixed with 500 mg of magenta toner in a turbula for 10 minutes at 90 rpm. The formulation did not mix as determined by visual inspection. Internal components showed some mixing, wall deposition and was clearly not homogenous.
1.3. LactoHale 230 (100 g) was mixed with 500 mg of magenta toner in a turbula for 2 minutes at 30 rpm. The formulation did not mix as determined by visual inspection.
1.4. LactoHale 230 (100 g) was mixed with 500 mg of magenta toner in a turbula for 10 minutes at 90 rpm. The formulation did not mix as determined by visual inspection. Internal components showed some mixing, wall deposition and was clearly not homogenous.
1.5. LactoHale 200 with a size range of 63-90 μm (100 g) was mixed with 500 mg of magenta toner in a turbula for 2 minutes at 30 rpm. The formulation mixed well as determined by visual inspection.
1.6. LactoHale 200 with a size range of 90-150 μm (100 g) was mixed with 500 mg of magenta toner in a turbula for 2 minutes at 30 rpm. The formulation mixed well as determined by visual inspection.
1.7. LactoHale 200 with a size range of 150-212 μm (100 g) was mixed with 500 mg of magenta toner in a turbula for 2 minutes at 30 rpm. The formulation mixed well as determined by visual inspection.

The results above demonstrate that finer grades of lactose (i.e. Sorbalac 400 and LactoHale230) did not mix with the toner

For the sake of comparison with API containing experiments, particle size analysis was conducted on the Hewlett Packard magenta toner used in the experiments. The data in table 1 confirms the suitability of this magenta toner for use as representative inhalable constituent, namely D₁₀≦6 μm, D₅₀≦7 μm and D₉₀≦10 μm. At no time is administration of magenta toner contemplated for administration to a human or animal subject.

TABLE 1
Particle size analysis by Malvern Morphologi G3 of Hewlett
Packard magenta toner (extracted from Laser Print cartridge)

| | Number distribution | | | Volume distribution | | |
|---|---|---|---|---|---|---|
| | D₁₀ | D₅₀ | D₉₀ | D₁₀ | D₅₀ | D₉₀ |
| Ave. | 4.35 | 5.88 | 7.72 | 5.094 | 6.748 | 8.604 |
| SD | 0.08 | 0.08 | 0.02 | 0.060 | 0.041 | 0.112 |
| % RSD | 1.93 | 1.43 | 0.27 | 1.17 | 0.600 | 1.296 |

### Example 2

2.1. Lactose (Sorbalac 400) 100 g was mixed with 500 mg of Magenta Printer Toner (Hewlett Packard, extracted from Laser Print cartridge) in a glass jar and clamped into a LabRAM (Resodyn). The resonance point was determined to vary depending on jar size, shape and powder load. Initial resonance was achieved at 61 Hz. The LabRAM was set to “Auto” mode to track and maintain the resonance of the jar and powder. This was determined to be 60.67 Hz. The intensity was increased from 15% to 45% which caused the acceleration to increase from 6 G to 50 G (roughly half the mixing power). This was timed for 2 minutes and stopped. The powder was visually inspected and found to have mixed well in contrast to formulation 1.1. mentioned above.
2.2. Lactose (Sorbalac 400) 100 g was mixed with 500 mg of Magenta Printer Toner (Hewlett Packard, extracted from Laser Print cartridge) in a glass jar and clamped into a LabRAM (Resodyn) under the following conditions using the same method as for 2.1. above to find the resonance point. Resonance was achieved at 60.64 Hz, the intensity was set at 45%, the acceleration was 50 G (roughly half the mixing power). This was timed for 10 minutes and stopped. The powder was visually inspected and found to have mixed well in contrast to formulation 1.2. mentioned above.

### Example 3

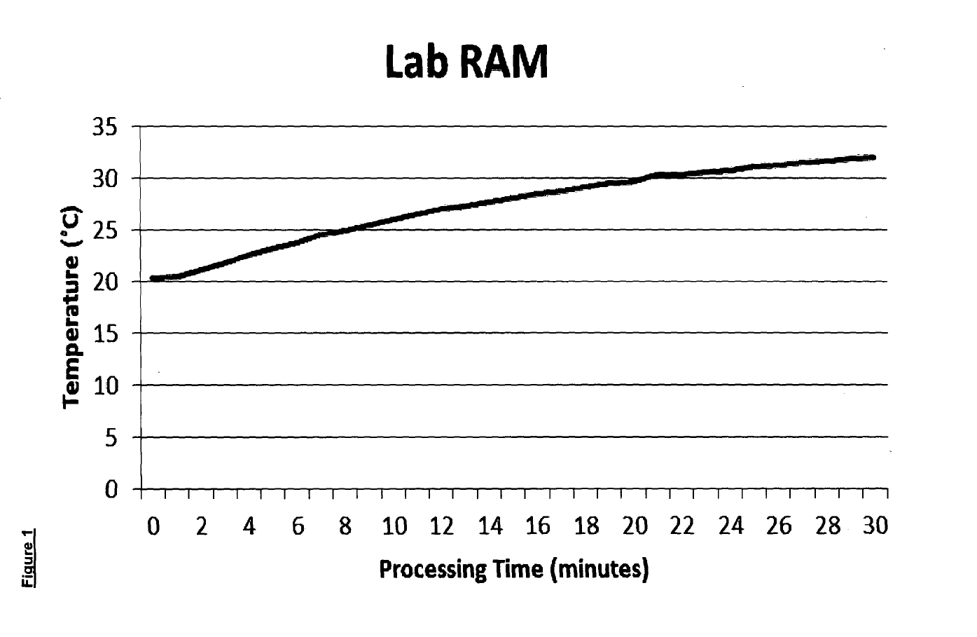

Lactose (LactoHale 200) 100 g, sieved 90-125 μm was mixed with 500 mg of Magenta Printer Toner (Hewlett Packard, extracted from Laser Print cartridge). A RS 206-3738 temperature probe was inserted through the jar's lid and into the powder. The processing conditions were as follows 60.35 Hz, 45% intensity and 31 G acceleration. The temperature in the powder was recorded every minute and is reported in FIG. 1 and Table 2 below:

TABLE 2
Temperature gain in a formulation after 0-30 minutes of
acoustic blending.

| Time | Temperature |
|---|---|
| (Minutes) | (° C.) |
| 0 | 20.3 |
| 1 | 20.5* |
| 2 | 21.1 |
| 3 | 21.8 |
| 4 | 22.6 |
| 5 | 23.2 |
| 6 | 23.8 |
| 7 | 24.5 |
| 8 | 25.0 |
| 9 | 25.5 |
| 10 | 26.0 |
| 11 | 26.5 |
| 12 | 27.0 |
| 13 | 27.3 |
| 14 | 27.7 |
| 15 | 28.1 |
| 16 | 28.5 |
| 17 | 28.8 |
| 18 | 29.1 |
| 19 | 29.5 |
| 20 | 29.7 |
| 21 | 30.3 |
| 22 | 30.4 |
| 23 | 30.6 |
| 24 | 30.8 |
| 25 | 31.1 |
| 26 | 31.3 |
| 27 | 31.5 |
| 28 | 31.7 |
| 29 | 31.9 |
| 30 | 32.1 |
| — | — |

*Powder and toner were well mixed as determined by visual inspection The formulation temperature rise following 30 minutes of processing was 11.8° C. For comparison the temperature gain for the same formulation in a commercially available high sheer mixer was as follows:

TABLE 3
Temperature gain for commercially available high sheer mixer
(TRV, manufactured by GEA)

| Speed | | Time | Temperature | Time | Temperature |
|---|---|---|---|---|---|
| (rpm) | Cooling | (Minutes) | (° C.) | (Minutes) | (° C.) |
| 800 | No | 0 | 19.9 | 20 | 45.8 |
| 800 | Yes | 0 | 18.5 | 20 | 26.5 |
| 1000 | No | 0 | 23.9 | 20 | 61.6 |
| 1000 | Yes | 0 | 17.5 | 20 | 31.4 |
| 1500 | No | 0 | 25.0 | 17** | 88.8 |
| 1500 | Yes | 0 | 16.7 | 20 | 43.5 |

** Due to the rapid increase in temperature, the temperature increase assessment was made early at 17 minutes.

The 11.8° C. temperature rise following 30 minutes of processing with the LabRAM was comparable with the mid processing speed (1000 rpm) on the commercially available high speed mixer (TRV) with chiller unit attached.

With respect to temperature gain, it is noteworthy that 30 minutes of blending using the resonant acoustic mixer (LabRAM) was equivalent to 20 minutes on the TRV. This demonstrates the ability of a resonant acoustic mixer (LabRAM) to process for longer periods without experiencing adverse effects due to temperature increases.

### Example 4

The following experiments were conducted to establish how long it took to achieve a homogenous blend using the LabRAM. Lactose (LH200 with 12% fines) was added to magenta toner (Hewlett Packard, extracted from Laser Print cartridge). A RS 206-3738 temperature probe was inserted through the jar's lid and into the powder. A frequency of 60.52 Hz was set for each blend. Uniformity of the formulation blend was determined by visual inspection. As with all experiments, a uniform pink colour was indicative of a uniform blend.

TABLE 4
Investigation into the relationship between
mixing intensity and acceleration.

| | | | | Accel- | Time | | |
|---|---|---|---|---|---|---|---|
| Blend | Lactose | Toner | Intensity | eration | (Min | Start | End |
| Number | (g) | (g) | (% power) | (G) | sec) | (° C.) | (° C.) |
| 1 | 100.32 | 0.5169 | 15 | 16 | 8′ 11″ | 18.2 | 19.0 |
| 2 | 100.18 | 0.5031 | 25 | 26 | 4′ 41″ | 18.0 | 18.6 |
| 3 | 100.25 | 0.5058 | 34 | 36 | 3′ 40″ | 19.9 | 20.4 |
| 4 | 100.25 | 0.5149 | 45 | 46 | 1′ 55″ | 20.3 | 20.6 |
| 5 | 100.26 | 0.5057 | 55 | 56 | 1′ 10″ | 20.2 | 20.5 |
| 6 | 100.39 | 0.5007 | 65 | 66 | 0′ 36″ | 19.4 | 19.7 |
| 7 | 100.31 | 0.5000 | 75 | 76 | 0′ 14″ | 19.6 | 19.8 |
| 8 | 100.08 | 0.5028 | 85 | 86 | 0′ 10″ | 19.3 | 19.4 |
| 9 | 100.11 | 0.5021 | 95 | 96 | 0′ 10″ | 19.6 | 19.7 |

### Example 5

The following experiments were conducted to achieve a homogenous blend using the LabRAM. Lactose (LH200 with 12% fines) was added to magenta toner (Hewlett Packard, extracted from Laser Print cartridge). A RS 206-3738 temperature probe was inserted through the jar's lid and into the powder. A frequency of 60.52 Hz, Intensity of 45%, acceleration of 44 G and blend time of 10 minutes was set for each blend.

TABLE 5
Data showing a decrease in blend temperature with increasing magnesium
stearate content.

| | | | | | Temperature |
|---|---|---|---|---|---|
| | Lactose | MgSt | Start | End | Increase |
| Blend Number | (g) | (g) | (° C.) | (° C.) | (° C.) |
| 1 | 95.00 | 5.0020 | 17.0 | 20.4 | 3.4 |
| 2 | 95.99 | 4.0084 | 17.2 | 20.6 | 3.4 |
| 3 | 97.00 | 3.0015 | 17.4 | 20.7 | 3.3 |
| 4 | 98.00 | 2.0028 | 17.6 | 22.6 | 5.0 |
| 5 | 99.00 | 1.0057 | 17.6 | 23.0 | 5.4 |
| 6 | 99.51 | 0.4753 | 17.6 | 23.3 | 5.7 |

All blends mixed well as determined by visual inspection. It is noteworthy that formulations containing higher magnesium stearate content exhibited a lower rise in processing temperature. This is particularly notable when contrasted with a control formulation containing (without magnesium stearate) consisting of Lactose (LactoHale 200) 100 g, sieved 90-125 μm and mixed with 500 mg of Magenta Printer Toner (Hewlett Packard, extracted from Laser Print cartridge). The processing conditions were as follows 60.35 Hz, 45% intensity and 31 G acceleration. The initial blend temperature was 20.3° C. and the final blend temperature at 10 minutes was 26.0° C. resulting in a temperature increase of 5.7° C.

### Example 6

The following experiments were conducted to investigate the efficiency of the blending process when using various sieve fractions of lactose (LH200) using the LabRAM. Magenta toner (Hewlett Packard, extracted from Laser Print cartridge) was added to the formulation. A frequency of 60.45 Hz, Intensity of 45% and blend time of 2 minutes was set for each blend.

TABLE 6
Investigation of the variation of sieve fractions used within acoustically
blended formulations.

| Blend Number | Lactose | MgSt | Toner | Acceleration | |
|---|---|---|---|---|---|
| (Sieve) | (g) | (g) | (g) | (G) | Results |
| 1 | 95.00 | 5.0020 | 0.4937 | 48 | Mixed Well |
| 0-63 μm | | | | | |
| 2 | 95.99 | 4.0084 | 0.5091 | 41 | Mixed Well |
| 63-90 μm | | | | | |
| 3 | 97.00 | 3.0015 | 0.5150 | 37 | Mixed Well |
| 90-125 μm | | | | | |
| 4 | 98.00 | 2.0028 | 0.5129 | 36 | Mixed Well |
| 125-150 μm | | | | | |
| 5 | 99.00 | 1.0057 | 0.4977 | 34 | Mixed Well |
| 150-212 μm | | | | | |

All the blends appeared homogeneous within 1 minute as determined by visual inspection. Different size fractions of LH200 result in different acceleration values when processed with the same mass frequency and intensity.

### Example 7

Processing wet-agglomerated lactose (Prismalac 355-600 μm) in a Diosna mixer resulted in a dramatic reduction is particle size suggesting that some lactose grades are sensitive to shear forces.

An investigation was conducted into the effect of acoustic mixing on shear sensitive powder. Prismalac (355-600 μm) 101.23 g was processed in a LabRAM acoustic mixer under the following conditions 45% intensity, 60.73 Hz, Acceleration of 42 G for 10 minutes.

Visual inspection showed that the powder agglomerates appeared unbroken.

TABLE 7
Volume distribution of unprocessed Prismalac (355-600 μm), Diosna
processed Prismalac (355-600 μm) and LabRAM processed Prismalac
(355-600 μm)

| | | |
|---|---|---|
| | Diosna | LabRAM |
| Unprocessed | Processed | processed |

Volume Distribution (μm)

| | | | |
|---|---|---|---|
| Min. | 12.50 | 12.43 | 12.43 |
| Max. | 777.33 | 279.15 | 786.51 |
| D₁₀ | 454.2 | 85.63 | 500.6 |
| D₅₀ | 570.7 | 153.7 | 551.1 |
| D₉₀ | 684.0 | 216.2 | 776.0 |

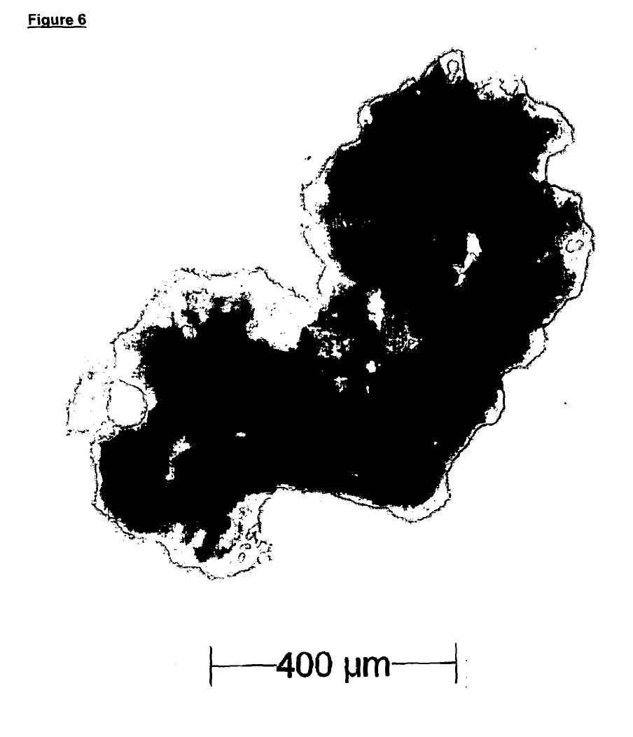

The particle size distribution of the LabRAM processed Prismalac (355-600 μm) closely matches that of the unprocessed material. Conversely the Diosna processed material shows a smaller particle size range. The morphologi G3 image (FIG. 6) of the LabRAM processed material shows that the agglomerate structure has not been broken down. These results demonstrate that it is possible to process shear sensitive powder using an acoustic mixer without adversely affecting the particle size distribution of shear sensitive particles.

### Example 8

Examples disclosed herein demonstrate that the Turbula does not adequately process certain types of lactose, specifically Lactohale 230 (d₁₀=1.4 μm, d₅₀=8.5 μm, d₉₀=24 μm).

Two batches of Lactohale 230 (80.05 g and 80.17 g) with Magenta Printer Toner (Hewlett Packard, extracted from Laser Print cartridge) (0.5181 g and 0.5101 g) in a glass jar and clamped into a LabRAM (Resodyn). The frequency was set at 60.67 Hz. The intensity was set at 45%.

The samples were processed at an acceleration of 47 G for 2 minutes and 10 minutes.

The cohesive powder was visually inspected and found to have mixed well in 2 minutes with no segregation occurring at longer processing times (e.g. up to 10 minutes).

### Example 9

Batches of Lactohale 300 were mixed with Magenta Printer Toner (Hewlett Packard, extracted from Laser Print cartridge) and blended using either Turbula or a LabRAM (Resodyn) using the following procedures:
1. Turbula, 2 minutes, 30 rpm.
2. Turbula, 10 minutes, 90 rpm.
3a. LabRAM, 2 minutes, 45% intensity, 60.68 Hz, 51 G.
3b. LabRAM, 10 minutes, 45% intensity, 60.68 Hz, 51 G.
4. LabRAM, 30 minutes, 15% intensity, 60.68 Hz, 17 G.

### Results:

1. Did not mix
2. Some internal mixing, unmixed lactose impacted on the walls
3a. Internal material mixed well, unmixed lactose impacted on the walls of the jar.
3b. Internal material mixed well, unmixed lactose impacted on the walls of the jar.
4. Material mixed well, homogeneous.

### Example 10

Magnesium stearate (MgSt) was passed through a 45 μm sieve and weighed into a glass jar. Milled apomorphine hydrochloride (Apo) and lactose (LH200) was weighed separately into the same glass jar and the components were mixed acoustically.

TABLE 8
Experimental design of apomorphine, magnesium stearate and
lactose containing formulations

| Blend | Lac- | | | | Accel- | Fre- | |
|---|---|---|---|---|---|---|---|
| Num- | tose | Apo | MgSt | Intensity | eration | quency | Time |
| ber | (g) | (g) | (g) | (% power) | (G) | (Hz) | (Mins) |
| 1 | 94.77 | 4.7513 | 0.4750 | 45 | 47 | 60.63 | 10 |
| 2 | 94.77 | 4.7526 | 0.4749 | 15 | 16 | 60.63 | 10 |

### Results (Content Uniformity):

TABLE 9
Content Uniformity of acoustically blended apomorphine, magnesium
stearate and lactose containing formulations

| Blend | | | | | |
|---|---|---|---|---|---|
| Number | Average | SD | % RSD | Min | Max |
| 1 | 100.72 | 1.83 | 1.82 | 98.61 | 104.39 |
| 2 | 96.83 | 19.92 | 20.57 | 84.37 | 148.7 |

The above formulations were processed at different parameters thereby demonstrating that a uniform blend can be obtained if the appropriate acoustic blending parameters are selected for the specific powder constituents.

Therefore 45% intensity methodology produced a blend with acceptable blend homogeneity (% RSD=1.82), whereas the 15% intensity methodology produced a blend with unacceptable blend homogeneity (% RSD=20.57).

A content uniformity of 20% could be avoided by diligent monitoring of the blend's content uniformity during acoustic blending to ensure the blend does not segregate.

The acceptable blend (“45% intensity methodology”) was assessed for aerosol performance using a GyroHaler (Trade Mark) at 60 L/min.

TABLE 10
Aerosol Performance (“45% intensity methodology”)

| | | Metered | Delivered | | | |
|---|---|---|---|---|---|---|
| | Shot Weight | Dose | Dose | FPM | | Mass |
| FSI | (mg) | (μg) | (μg) | (<5 μm) | FPF | Balance |
| 1 | 19.7 | 951.21 | 881.18 | 476.83 | 54.1 | 100.1 |
| 2 | 19.6 | 920.31 | 850.28 | 459.80 | 54.1 | 96.9 |
| 3 | 19.7 | 904.90 | 834.87 | 463.80 | 55.6 | 95.3 |
| Mean | 19.7 | 925.47 | 855.44 | 466.81 | 54.6 | 97.4 |
| SD | 0.1 | 23.58 | 23.58 | 8.9 | 0.8 | 2.5 |
| % RSD | 0.3 | 2.5 | 2.8 | 1.9 | 1.5 | 2.5 |

The “45% intensity methodology” produced an inhalable formulation with excellent aerosol performance from a GyroHaler (Trade Mark) inhaler device.

### Example 11

Lactose+toner. LabRAM operating parameters 60.35 Hz at 45% intensity. A uniform blend was achieved in 2 minutes with temperature gain of 0.8° C. After 30 minutes, the temperature rise was 11.8° C. In comparison a TRV (Blade mixer) had a temperature gain of 63.8 C when processing Lactose with no cooling jacket, operated at 1500 rpm for 17 minutes.

The TRV process with a cooling jacket, operated at 1500 rpm for 20 minutes gave a temperature gain of 26.8° C.

### Example 12

An agglomerated lactose (max. raw particle size 777 μm) was processed by Diosna (800 rpm for 2 minutes. The resultant particle size was 279 μm, showing that this material is sensitive to the action of paddles/rotors within a mixing vessel.

When this material was processed on the LabRAM (45% intensity and acceleration 42 G), 10 minutes at 60.73 Hz), there was no decrease in the particle size (max. 786 μm), showing that this acoustic mixing method is suitable for processing brittle/impact/shear sensitive material.

### Example 13

Apomorphine (4.75%), magnesium stearate (0.475%) and lactose (94.775%) was processed on the LabRAM (45% Intensity, 60.63 Hz, 47 G for 10 minutes) in one pot with no “sandwiching” i.e. lactose was put into the pot, followed by the apomorphine, followed by the magnesium stearate. A sandwiched blend would, for example, have a layer of lactose followed by the API and magnesium stearate followed by more lactose (or multiple layers formed in this manner). This gave a content uniformity % RSD of 1.82, showing that a homogenous blend is achievable. The aerodynamic performance (FPF=54.6% via an FSI) was excellent and comparable to standard Diosna blends of the same material.

Further blends were manufactured as above without the need to layer the constituent parts:

Fluticasone propionate (0.4%) and lactose (99.6%) was processed on the LabRAM (45% Intensity, 60.51 Hz, 46 G for 10 minutes) in one pot with no sandwiching of the ingredients. This gave a content uniformity % RSD of 4.06, showing that a homogenous blend is achievable. The aerodynamic performance (FPF=22.81% via an FSI) was comparable to standard Diosna blends of the same material.

Fluticasone propionate (4.0%) and lactose (96%) was processed on the LabRAM (45% Intensity, 60.51 Hz, 47 G for 10 minutes) in one pot with no ‘sandwiching’ of the ingredients. This gave a content uniformity % RSD of 5.35, showing that a homogenous blend is achievable. The aerodynamic performance (FPF=32.4% via an FSI) was comparable to standard Diosna blends of the same material.

Salmeterol xinafoate (0.5182%) and lactose (99.4188%) was processed on the LabRAM (45% Intensity, 60.50 Hz, 47 G for 10 minutes) in one pot with no “sandwiching” of the ingredients. This gave a content uniformity % RSD of 5.51, showing that a homogenous blend is achievable. The aerodynamic performance (FPF=18.38% via an FSI) was comparable to standard Diosna blends of the same material.

### Example 14

A blend of apomorphine 4.75% w/w and LH200 95.25 w/w was mixed in an acoustic mixer with an intensity of 45%, acceleration of 47 G and frequency of 60.63 Hz. This blend was mixed for 10 minutes.

TABLE 11
Content uniformity

| | Ave. | SD | % RSD | Min. | Max. |
|---|---|---|---|---|---|
| 45% intensity | 98.14 | 2.79 | 2.85 | 94.87 | 104.76 |
| (no magnesium stearate) | | | | | |

This acceptable blend was assessed for aerosol performance using a GyroHaler (Trade Mark) at 60 L/min.

TABLE 12
Aerosol performance

| | Shot | Metered | Delivered | FPM | |
|---|---|---|---|---|---|
| | Weight | Dose | Dose | <5 μm | FPF |
| FSI | (mg) | (μg) | (μg) | (μg) | (%) |
| 1 | 14.0 | 783.67 | 650.45 | 258.84 | 39.8 |
| 2 | 20.6 | 1049.15 | 915.92 | 296.08 | 32.3 |
| 3 | 19.8 | 992.86 | 859.63 | 304.80 | 35.5 |
| Mean | 18.1 | 941.89 | 808.67 | 286.57 | 35.9 |
| SD | 3.6 | 139.88 | 139.88 | 24.41 | 3.8 |
| % RSD | 19.9 | 14.9 | 17.3 | 8.5 | 10.5 |

This alternate methodology still produced an inhalable formulation with acceptable aerosol performance from a GyroHaler (Trade Mark) inhaler device. When contrasted with Example 10 (above) the beneficial effect of magnesium stearate for improving aerosol performance is clearly evident, namely 54.6% FPF (with magnesium stearate) compared with 35.9% FPF (without magnesium stearate).

### Example 15

The “15% intensity methodology” blend mentioned above (Example 10) was subjected to a further intensity of 45%, acceleration of 47 G and frequency of 60.63 Hz and blended for 10 minutes.

TABLE 13
Content uniformity

| | Ave. | SD | % RSD | Min. | Max. |
|---|---|---|---|---|---|
| 15% initial | 100.17 | 2.19 | 2.18 | 97.28 | 104.72 |
| intensity reworked | | | | | |
| with 45% intensity | | | | | |
| (with magnesium | | | | | |
| stearate) | | | | | |

This acceptable blend, as determined by content uniformity, was assessed for aerosol performance using a GyroHaler (Trade Mark) at 60 L/min.

TABLE 14
Aerosol performance

| | Shot | Metered | Delivered | FPM | |
|---|---|---|---|---|---|
| | Weight | Dose | Dose | <5 μm | FPF |
| FSI | (mg) | (μg) | (μg) | (μg) | (%) |
| 1 | 19.70 | 896.02 | 831.76 | 491.71 | 59.1 |
| 2 | 20.30 | 885.82 | 821.56 | 475.07 | 57.8 |
| 3 | 19.40 | 856.11 | 791.85 | 434.40 | 54.9 |
| Mean | 19.8 | 879.32 | 815.06 | 467.06 | 57.3 |
| SD | 0.5 | 20.7 | 20.7 | 29.5 | 2.2 |
| % RSD | 2.3 | 2.4 | 2.5 | 6.3 | 3.8 |

This alternate methodology now produced an inhalable formulation with acceptable aerosol performance from a GyroHaler (Trade Mark) inhaler device despite initially producing unacceptable results from 15% intensity acoustic blending.

This methodology also demonstrates that it is possible for the resonance blending to be interrupted whilst, for example, an alternate acoustic blending methodology is selected when unacceptable content uniformity is obtained (Example 10, Formulation 2 using 15% Intensity). Upon completion of the content uniformity assessment, and selection of a new set of parameters, resonance blending may be resumed.

### Example 16

Bulk glycopyrrolate (2.388 μm (D₁₀), 23.926 μm (D₅₀), and 188.69 μm (D₉₀)) was micronized using a jet-mill (AS50 jet mill) (0.942 μm (D₁₀), 2.534 μm (D₅₀), and 5.817 μm (D₉₀)) producing 20 g micronized glycopyrrolate powder. The jet-mill conditions were 5 bar venturi and 3 bar grind pressure with feed rate of 2 g·min⁻¹. The resulting formulation was split into two separate samples. One sample was the control which was placed in a 120 mL ointment jar under atmospheric environmental conditions. This sample quickly congealed and a particle size distribution proved difficult.

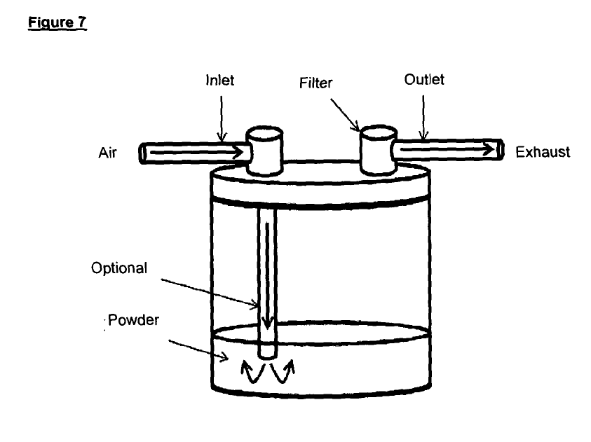

The remaining micronised glycopyrrolate sample was conditioned using the LabRAM by exposure to humid conditions (60-80% RH) at room temperature (approximately 22° C.) whilst applying low frequency (approximately 60 Hz), high-intensity acoustic energy (10% or 30% intensity) by use of a conditioning cell (FIG. 7). The cell incorporated a purging system which, using an air pump, constantly flushed the humidified air through the cell. The conditioning duration was not more than 10 minutes. The glycopyrrolate conditioned sample was stored in a 120 mL ointment jar under atmospheric environmental conditions.

The conditioned glycopyrrolate samples were analysed using the Malvern Mastersizer 2000 at 0, 24 and 168 hrs (1 week) post-conditioning. A summary of the particle size results is presented in Table 15.

TABLE 15
Particle size analysis 0, 24 and 168 hrs post conditioning

| | | PSD | | |
|---|---|---|---|---|
| | | (0, 24 or 168 hrs post-conditioning) | | |
| Sample Type | μm | 0 | 24 | 168 |
| LabRAM @ 10% ᵢₙ | D₁₀ | 0.902 | 0.934 | 0.904 |
| (60-80% RH) | D₅₀ | 2.619 | 2.725 | 2.601 |
| | D₉₀ | 5.817 | 6.119 | 5.348 |
| LabRAM @ 30% ᵢₙ | D₁₀ | 0.902 | 0.895 | 0.911 |
| (60-80% RH) | D₅₀ | 2.619 | 2.664 | 2.592 |
| | D₉₀ | 5.817 | 5.981 | 5.139 |

### Example 17

The conditioned glycopyrrolate (produced using either 0 hrs, 24 hrs or 168 hrs post-conditioning) may then be further partitioned into sub-samples. The data range (presented above) of 0 hrs to 168 hrs demonstrates that conditioned glycopyrrolate produced within this range is eminently suitable for use in an inhalable formulation. These sub-samples of conditioned glycopyrrolate may then acoustically blended at 45% intensity and 60.52 Hz for 10 minutes with either 1% or 0.5% or 0.1% (w/w of final formulation) magnesium stearate. Once homogenously blended in the acoustic mixer, lactose (LH200, LH300 or ML001 98% (w/w of final formulation)) may then be added and then acoustically blended at 45% intensity and 60.52 Hz for 10 minutes or until a coefficient of variation of less than 5% has been achieved.

Alternatively, the blend components glycopyrrolate (preferably glycopyrronium bromide), magnesium stearate, lactose and, optionally indacaterol maleate, may be blended together without the need for serial addition of formulation constituent parts. Blending until a coefficient of variation of less than 5% has been achieved.

Once a coefficient of variation of less than 5% has been achieved for either the single API or both APIs, the homogenous blend may then be filled into a powder receptacle, for example either hydroxypropyl methylcellulose (HPMC) capsule or foil blisters (foil strip or foil pack) for use in an inhaler device.

The stability of the pharmaceutical composition comprising the pharmaceutically active material mentioned above can be determined by a consistent fine particle fraction of at least 30%, for a period of at least 1 month, preferably 6 months, preferably 12 months, more preferably 18 months or most preferably 24 months.

1-59. (canceled)

60. A method for making a pharmaceutical composition for pulmonary administration, the method comprising a step in which an inhalable pharmaceutically active material is acoustically blended in a resonant acoustic blender, wherein the pharmaceutical composition is for localised pulmonary administration, preferably wherein the active is for localised effect, alternatively wherein the active is for systemic effect.

61. A method according to claim 60 wherein the acoustic blending is conducted at from 5 Hz to 1,000 Hz, more preferably 60 Hz to 75 Hz, most preferably from about 60 to 61 Hz.

62. A method according to claim 60 wherein the acoustic blending is conducted for at least 1 minute, for at least 2 minutes, for at least 3 minutes

63. A method according to claim 60 wherein the pharmaceutical composition comprises an excipient material.

64. A method according to claim 60 wherein the pharmaceutical composition further comprises additive material.

65. A method according to claim 60 wherein the pharmaceutically active material is particulate.

66. A method according to claim 60, wherein the pharmaceutically active material is selected from a long-acting muscarinic antagonist and/or long-acting beta-adrenoceptor agonist and/or an inhaled corticosteroid.

67. A method according to claim 60, wherein the pharmaceutically active material is selected from budesonide, formoterol fumarate, glycopyrronium bromide, indacaterol maleate, umeclidinium bromide, vilanterol trifenatate, tiotropium bromide, salmeterol xinafoate or fluticasone propionate.

68. A method according to claim 60, wherein the pharmaceutically active material is glycopyrronium bromide and indacaterol maleate

69. A method according to claim 60, wherein the pharmaceutically active material is fluticasone furoate and vilanterol trifenatate.

70. A method according to claim 60, wherein the pharmaceutically active material is tiotropium bromide.

71. A method according to claim 60, wherein the pharmaceutically active material is umeclidinium bromide and vilanterol trifenatate.

72. A method according to claim 60, wherein the pharmaceutically active material is glycopyrronium bromide.

73. A method according to claim 63 in which the excipient is lactose.

74. A method according to claim 73 wherein the excipient is alpha-lactose monohydrate, preferably wherein in the D₁₀≦250 μm, D₅₀≦500 μm and D₉₀≦800 μm, more preferably wherein in the D₁₀≦5-15 μm, D₅₀≦60-80 μm and D₉₀≦120-160 μm, most preferably D₁₀≦15 μm, D₅₀≦80 μm and D₉₀≦160 μm.

75. A method as claimed in claim 64 wherein the additive material is particulate.

76. A method as claimed in claim 75, in which the additive material comprises an amino acid.

77. A method as claimed in claim 75, in which the additive material comprises a phospholipid.

78. A method as claimed in claim 75, in which the additive material comprises a metal stearate.

79. A method as claimed in claim 78, wherein the metal stearate is either magnesium stearate or calcium stearate, preferably wherein the additive is magnesium stearate.

80. A method according to claim 60, wherein the acoustic blending step is carried out in the presence of a liquid.

81. A method as claimed in claim 80, wherein the liquid comprises a propellant suitable for use in a pressurised metered dose inhaler device.

82. A method according to claim 60, wherein the composition is conditioned during the acoustic blending.

83. A method of claim 60, wherein the active material is conditioned during the acoustic blending.

84. A method of claim 83, wherein the active material is conditioned by an elevated level of relative humidity as compared to ambient conditions.

85. A method of claim 83, wherein the active material is conditioned by increasing the relative humidity over time to about 60-80% RH, preferably to about 75%.

86. A method of claim 83, wherein the active material is conditioned at a minimum temperature, wherein the minimum temperature is 20° C.

87. A method of claim 83, wherein the pharmaceutically active material is selected from a long-acting muscarinic antagonist, long-acting beta-adrenoceptor agonist and/or inhaled corticosteroid.

88. A method according to claim 87, wherein the pharmaceutically active material is selected from budesonide, formoterol fumarate, glycopyrronium bromide, indacaterol maleate, umeclidinium bromide, vilanterol trifenatate, tiotropium bromide, salmeterol xinafoate or fluticasone propionate.

89. A method according to claim 87, wherein the pharmaceutically active material is glycopyrronium bromide and indacaterol maleate.

90. A method according to claim 87, wherein the pharmaceutically active material is fluticasone furoate and vilanterol trifenatate.

91. A method according to claim 87, wherein the pharmaceutically active material is tiotropium bromide.

92. A method according to claim 87, wherein the pharmaceutically active material is umeclidinium bromide and vilanterol trifenatate.

93. A method according to claim 87, wherein the pharmaceutically active material is glycopyrronium bromide.

94. The method of claim 60, wherein the active material is micronised prior to acoustic blending.

95. The method of claim 94, wherein the micronisation is by impact milling or jet milling, preferably air-jet milling.

96. A method according to claim 60, wherein after acoustic blending the active ingredient is packaged into a receptacle or delivery device.

97. A pharmaceutical composition comprising an active material, obtained using the method of claim 60.

98. A pharmaceutical composition according to claim 97 further comprising an excipient material and optionally an additive material.

99. A pharmaceutical composition according to claim 98 wherein the excipient is lactose, preferably alpha-lactose monohydrate.

100. An inhaler device comprising a composition obtained using the method claimed in any one of claim 60.

101. A pharmaceutical composition obtained by the method of claim 60 for use in medicine.

102. A method for preparing composite active particles for use in a pharmaceutical composition for pulmonary administration, the method comprising a step in which particles of active material are acoustically blended in the presence of particles of an additive material which is suitable for the promotion of the dispersal of the composite active particles upon actuation of an inhaler.

103. A method for making a pharmaceutical composition for pulmonary administration, the method comprising a step in which particles of the pharmaceutical composition are acoustically blended together to create composite particles, wherein pharmaceutically active material is acoustically blended in the presence of particles of additive material so that the additive material coats the active particles, wherein the additive material comprises an amino acid, a metal stearate, or a phospholipid.

104. A method for making a pharmaceutical composition, the method comprising a step in which an inhalable pharmaceutically active material is acoustically blended with excipient material, wherein the acoustic frequency operating range is from 5 Hz to about 1,000 Hz for a period for at least 2 minutes, preferably, wherein the excipient material comprises lactose.

105. Use of a resonant acoustic blender for the preparation of an inhalable pharmaceutical composition.

106. The use according to claim 105 in which the composition is an inhalable composition for treatment of respiratory diseases.
